(19) United States
(12) Patent Application Publication
(10) Pub. No.: US 2024/0150420 A1
(43) Pub. Date: May 9, 2024

(54) NUCLEIC ACID ENCODING HUMAN HGF AND USE THEREOF

(71) Applicant: Peking University, Beijing (CN)

(72) Inventors: Kai Wang, Beijing (CN); Jiayan Lang, Beijing (CN); Shaoli Liu, Beijing (CN); Zihang Pan, Beijing (CN); Weijing Kong, Beijing (CN); Andong Liu, Beijing (CN)

(21) Appl. No.: 18/182,413

(22) Filed: Mar. 13, 2023

(30) Foreign Application Priority Data
Oct. 31, 2022 (CN) 202211345337.7

(51) Int. Cl. C07K 14/475; C12N 15/63

(52) U.S. Cl. CPC C07K 14/4753; C12N 15/63

(57) ABSTRACT

The present disclosure relates to a nucleic acid encoding human HGF and use thereof. The nucleic acid comprises one or more open reading frames (ORFs). The ORF nucleic acid sequence is at least 80%, at least 85%, at least 90%, at least 95%, at least 96%, at least 97%, at least 97%, at least 98%, at least 99% or 100% identical to the nucleic acid sequence of SEQ ID NOs: 21-31. The present disclosure provides a nucleic acid encoding human HGF, as well as a nucleic acid construct, a vector, a cell and a drug which comprise the nucleic acid. The nucleic acid has a protein expression level superior to that of a natural sequence.

### CROSS REFERENCE TO RELATED SEQUENCE LISTING

This application contains a computer readable form of a Sequence Listing, the name of the file being “Sequence Listing”, created on 17 Jul. 2023 and electronically submitted via Patent Center on 17 Jul. 2023. The size of the xml file is 69,883 bytes and the file is incorporated herein by reference.

### CROSS REFERENCE TO RELATED APPLICATION

The present application claims the benefit of Chinese application No. 202211345337.7 filed with the CNIPA on 31 Oct. 2022, the entire disclosure of which is incorporated herein by reference.

### TECHNICAL FIELD

The present disclosure belongs to the technical field of biology, and particularly relates to a nucleic acid encoding human HGF and use thereof.

### BACKGROUND

A hepatocyte growth factor (HGF) is a multifunctional cytokine. An HGF/c-Met system functions involve survival, differentiation, proliferation, inflammation resistance and fibrosis resistance of cells, and play an important role in the aspects of embryogenesis, wound healing, angiogenesis, tissue organ regeneration, morphogenesis, carcinogenesis and the like.

A phenomenon that the same amino acid has two or more codons is called degeneracy of codons. Synonymous codons are usually different at the third base. The presence of synonymous codons allows one protein or polypeptide sequence to have multiple different nucleic acid encoding sequences, whereas the stabilities and protein expression efficiencies of these different nucleic acid sequences in cells have significant differences. Searching for a sequence design with a better effect is one of the key research contents of nucleic acid drug development.

### SUMMARY

In order to solve the problems existing in the prior art, the present disclosure provides a nucleic acid encoding human HGF, as well as a nucleic acid construct, a vector, a cell and a drug which comprise the nucleic acid. The nucleic acid has a protein expression level superior to that of a natural sequence.

The objective of the present disclosure is to provide a nucleic acid.

The objective of the present disclosure is to provide a construct comprising the above nucleic acid.

Another objective of the present disclosure is to provide a vector, a cell and a drug which comprise the nucleic acid.

Provided is a nucleic acid according to specific embodiments of the present disclosure, the nucleic acid comprising one or more open reading frames (ORFs), wherein the ORF nucleic acid sequence is at least 80%, at least 85%, at least 90%, at least 95%, at least 96%, at least 97%, at least 97%, at least 98%, at least 99% or 100% identical to the nucleic acid sequence of SEQ ID NOs: 21-31. The nucleic acid has a protein expression level superior to that of a natural sequence.

Further, the ORF nucleic acid sequence is selected from SEQ ID NOs: 21-31, or a transcribed RNA sequence thereof.

Preferably, the nucleic acid further comprises a 5′cap.

Preferably, the 5′cap is selected from m7G5′ppp5′Np, m7G5′ppp5′NmpNp and m7G5′ppp5′NmpNmpNp.

Preferably, the nucleic acid further comprises 5′UTR.

Preferably, the 5′UTR comprises one sequence selected from SEQ ID NOs: 1-8, or a combination thereof.

Preferably, the nucleic acid further comprises 3′UTR.

Preferably, the 3′UTR comprises one sequence selected from SEQ ID NOs: 9-17, or a combination thereof.

Preferably, the nucleic acid further comprises a poly-A region comprising 70-150 nucleotides in length.

Preferably, the poly-A region comprises the sequence set forth in SEQ ID NO: 18 or 19.

Preferably, the nucleic acid comprises a sequence selected from SEQ ID NOs: 32, 33, 34, 35, 56, 57, 58, 60, 61, 63 and 65, or a transcribed RNA sequence thereof.

Preferably, the nucleic acid comprises one or more modified nucleosides selected from pseudouridine, N1-methyl-pseudouridine and 5-methylcytidine.

Preferably, the nucleic acid is DNA or mRNA.

Preferably, the protein expression level of the nucleic acid has a protein expression level that is at least 10%, at least 20%, at least 30%, at least 40% or at least 50% higher than that of a natural sequence.

Provided is a vector comprising the nucleic acid.

Provided is a cell comprising the nucleic acid.

Provided is a pharmaceutical composition comprising the nucleic acid.

Provided is a method for expressing a polypeptide in a mammal. By the method, the cell is in contact with the nucleic acid or the pharmaceutical composition.

Provided is use of the nucleic acid or the pharmaceutical composition in preparation of a drug for treating or preventing a disease. Preferably, the disease is a disease with insufficient HGF expression level.

Unless otherwise stated, the terms have general meaning.

In the present disclosure, “polypeptide” or “protein” refers to a polymer formed by connection of amino acids via a peptide bond. The amino acid is selected from 20 natural amino acids or other non-natural amino acids. The 20 natural amino acids refer to glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine and histidine.

The nucleotide is a kind of compounds consisting of three substances, that is, purine or pyrimidine bases, ribose or deoxyribose and phosphoric acid.

“Nucleic acid” refers to a polymer formed through connection of nucleotides via a 3′,5′-phosphate diester bond. The nucleic acid is a single-stranded or double-stranded deoxyribonucleic acid (DNA) or ribonucleic acid (RNA) molecule and a heterozygous molecule thereof. The examples of the nucleic acid molecule include but are not limited to messenger RNA (mRNA), microRNA (miRNA), small interfering RNA (siRNA), self-amplifying RNA (saRNA) and antisense oligonucleotide (ASO). Preferably, the nucleic acid is mRNA.

The nucleic acid can be further chemically modified. Preferably, the chemical modification of mRNA is selected from one of pseudouridine, N1-methyl-pseudouridine, 5-methoxyuridine, 5-methylcytosine or a combination thereof.

The open reading frame (ORF) refers to a base sequence that is located between a starting codon and a termination codon and encodes a protein. The mRNA molecule comprises ORF, and optionally further comprises an expression regulatory sequence. The typical expression regulatory sequence includes but is not limited to 5′cap, 5′ untranslated region (5′UTR), 3′ untranslated region (3′UTR), a polyadenylate sequence (polyA) and a miRNA binding site.

The mRNA 5′cap can be obtained by connecting guanosine with the 5′ end of mRNA via a 5′-5 phosphate bond. The 5′ guanosine can be further modified, for example, is methylated to generate N7-methyl-guanosine residue. The nucleotide at position 1 or 2 of the 5′ end of mRNA can be further modified, for example, a ribose moiety undergoes 2′-O-methylation. The examples of 5′cap include but are not limited to m7G5′ppp5′Np (type O), m7G5′ppp5′NmpNp (type I) and m7G5′ppp5′NmpNmpNp (type II). The 5′ end cap structure of mRNA provides a single for a ribosome to identify mRNA, and assists in binding of the ribosome to mRNA. The cap structure can increase the stability of mRNA and protect mRNA from being degraded by 5′→3′ exonuclease. In some embodiments, the cap is not present.

The untranslated region (UTR) can be transcribed but not translated. The 5′UTR comprises a sequence from a transcription starting site to a starting codon, but does not comprise the starting codon. The 3′UTR comprises a sequence from a termination codon to a transcription termination signal but does not comprise the termination codon. The examples of UTR include but are not limited to sequences listed in Table 1.

TABLE 1
17 different sequences Of UTR

| | | SEQ ID |
|---|---|---|
| Number | Sequence | NO. |
| 5′UTR-1 | AGGAAATAAGAGAGAAAAGAAGAGTAAGAAGAAATA | 1 |
| | TAAGAGCCACC | |
| 5′UTR-2 | GGGCGAACTAGTACTCTTCTGGTCCCCACAGACTCGC | 2 |
| | CACC | |
| 5′UTR-3 | TCTCAACACAACATATACAAAACAAACGAATCTCAAG | 3 |
| | CAATCAAGCATTCTACTTCTATTGCAGCAATTTAAATC | |
| | ATTTCTTTTAAAGCAAAAGCAATTTTCTGAAAATTTTC | |
| | ACCATTTACGAACGATAGC | |
| 5′UTR-4 | AAGTTGAAAGTCGCCGCTGACAGTTGTGACCAGGAT | 4 |
| | CGGACAGGTGAAC | |
| 5′UTR-5 | ACATTTGCTTCTGACACAACTGTGTTCACTAGCAACC | 5 |
| | TCAAACAGACACC | |
| 5′UTR-6 | ACTCTTCTGGTCCCCACAGACTCAGAGAGAACCCACC | 6 |
| 5′UTR-7 | ACTCCCCGAACCACTCAGGGTCCTGTGGACAGCTCAC | 7 |
| | CTAGCTGCA | |
| 5′UTR-8 | ATAAACGCTCAACTTTGGCC | 8 |
| 3′UTR-1 | GCTGGAGCCTCGGTGGCCATGCTTCTTGCCCCTTGGG | 9 |
| | CCTCCCCCCAGCCCCTCCTCCCCTTCCTGCACCCGTAC | |
| | CCCCGTGGTCTTTGAATAAAGTCTGA | |
| 3′UTR-2 | GCTCGCTTTCTTGCTGTCCAATTTCTATTAAAGGTTCC | 10 |
| | TTTGTTCCCTAAGTCCAACTACTAAACTGGGGGATATT | |
| | ATGAAGGGCCTTGAGCATCTGGATTCTGCCTAATAAA | |
| | AAACATTTATTTTCATTGCGCTCGCTTTCTTGCTGTCC | |
| | AATTTCTATTAAAGGTTCCTTTGTTCCCTAAGTCCAAC | |
| | TACTAAACTGGGGGATATTATGAAGGGCCTTGAGCATC | |
| | TGGATTCTGCCTAATAAAAAACATTTATTTTCATTGC | |
| 3′UTR-3 | CTGGTACTGCATGCACGCAATGCTAGCTGCCCCTTTCC | 11 |
| | CGTCCTGGGTACCCCGAGTCTCCCCCGACCTCGGGTC | |
| | CCAGGTATGCTCCCACCTCCACCTGCCCCACTCACCA | |
| | CCTCTGCTAGTTCCAGACACCTCCCAAGCACGCAGCA | |
| | ATGCAGCTCAAAACGCTTAGCCTAGCCACACCCCCAC | |
| | GGGAAACAGCAGTGATTAACCTTTAGCAATAAACGAA | |
| | AGTTTAACTAAGCTATACTAACCCCAGGGTTGGTCAAT | |
| | TTCGTGCCAGCCACACC | |
| 3′UTR-4 | GCTCGCTTTCTTGCTGTCCAATTTCTATTAAAGGTTCC | 12 |
| | TTTGTTCCCTAAGTCCAACTACTAAACTGGGGGATATT | |
| | ATGAAGGGCCTTGAGCATCTGGATTCTGCCTAATAAA | |
| | AAACATTTATTTTCATTGC | |
| 3′UTR-5 | GCAGCTCGACGCCCGTTCGCTTGGTTCTGCCTGATTA | 13 |
| | CCATCCAGTCGGGTGTGGGCCGTTACCACACCGGTGA | |
| | ATAGTTACCTGAAGCTTGGTCAAACCTGGAACATGTT | |
| | GGTTCCACACCTTCATATCTCAGGCAGCAGAAAAACA | |
| | TGAAGGATAAGTGAAACGCCTGCACTGATAAATCAAA | |
| | GAAGAGGGTAAAATGAAGGTCATATTTTTTCTGAAAA | |
| | TGCATAAATAATCTTTTAAAAATATATATACATACTGT | |
| | ATAGAGAGAGAGAGCGGTCCATGGCATTATTGCTGCTG | |
| | AGTGACAGCTTAAGTTCAACCCAGGACAGGACTGCTGA | |
| | TCCAGCTGTGCTGAATCCATTTTTATTGTATTACCAGA | |
| | AATACACGTTACAGTAATGTTTTTACAATATAAACATG | |
| | AGTAGTTGTGTATTTTCTAGAAGTTTACCGCCTCTTGT | |
| | TATTTGACATTAGCTTTCTTTCTCATTTATTTTCTTGT | |
| | AAATAAATCTCTTGTGCTC | |
| 3′UTR-6 | GCTCGCTTTCTTGCTGTCCAATTTCTATTAAAGGTTCC | 14 |
| | TTTGTTCCCTAAGTCCAACTACTAAACTGGGGGATATT | |
| | ATGAAGGGCCTTGAGCATCTGGATTCTGCCTAATAAA | |
| | AAACATTTATTTTCATTGCAA | |
| 3′UTR-7 | GCTGGAGCCTCGGTGGCCATGCTTCTTGCCCCTTGGG | 15 |
| | CCTCCCCCCAGCCCCTCCTCCCCTTCCTGCACCCGTA | |
| | CCCCCGTGGTCTTTGAATAAAGTCTGAGTGGGCGGCA | |
| 3′UTR-8 | CTGCCCGGGTGGCATCCCTGTGACCCCTCCCCAGTGC | 16 |
| | CTCTCCTGGCCCTGGAAGTTGCCACTCCAGTGCCCAC | |
| | CAGCCTTGTCCTAATAAAATTAAGTTGCATCAT | |
| 3′UTR-9 | ACCAGCCTCAAGAACACCCGAATGGAGTCTCTAAGCT | 17 |
| | ACATAATACCAACTTACACTTTACAAAATGTTGTCCCC | |
| | CAAAATGTAGCCATTCGTATCTGCTCCTAATAAAAAGA | |
| | AAGTTTCTTCACA | |

The polyadenylic acid (poly-A) protects mRNA from being degraded by 5′→3′ exonuclease and increases the stability of mRNA itself. Poly-A comprises 100-250 nucleotides in length, preferably 100-150 nucleotides in length. The examples of poly-A include but are not limited to sequences listed in Table 2.

TABLE 2
TwO different sequences Of pOly-A

| | | SEQ |
|---|---|---|
| | | ID |
| Number | Sequence | NO. |
| pOly | AAAAAAAAAAAAAAAAAAAAAAAAAAAAAAGCATA | 18 |
| A-1 | TGACTAAAAAAAAAAAAAAAAAAAAAAAAAAAAAA | |
| | AAAAAAAAAAAAAAAAAAAAAAAAAAAAAAAAAA | |
| | AAAAAA | |
| pOly | AAAAAAAAAAAAAAAAAAAAAAAAAAAAAAAAAA | 19 |
| A-2 | AAAAAAAAAAAAAAAAAAAAAAAAAAAAAAAAAA | |
| | AAAAAAAAAAAAAAAAAAAAAAAAAAAAAAAAAA | |
| | AAAAAAAAAAAAAAAAAA | |

miRNA is a kind of endogenous non-encoding RNA comprising 19-25 nucleotides in length, which can identify and bind the miRNA binding site of a nucleic acid molecule, reduce the stability of the nucleic acid molecule or inhibit the translation process and further down regulate the gene expression level. The miRNA binding site is removed from a natural nucleic acid sequence, which can increase the protein expression; alternatively, one or more miRNA binding sites are added in the nucleic acid sequence to reduce the protein expression. Preferably, a miR-122 binding site is added in the nucleic molecule, thereby inhibiting the expression of a target gene in liver.

“Composition” refers to any product comprising specified amounts of various specified components.

“Pharmaceutical composition” refers to a composition comprising active ingredients, which can further comprise pharmaceutically acceptable excipients and other optional treatment components. The pharmaceutical composition of the present invention comprises pharmaceutical compositions which are suitable for oral, rectal, local and parenteral administration (including subcutaneous, intramuscular and intravenous administration). The pharmaceutical composition of the present invention can be conveniently present in a unit dosage form known in the art and prepared by using any preparation method known in the field of pharmacy.

Compared with the prior art, the present invention has the beneficial effects:
1. The present invention provides the mRNA encoding human HGF, which has lower folding free energy (MFE, predicted minimum folding energy) than that of a natural sequence, and the mRNA molecule is more stable.
2. The present invention provides a nucleic acid encoding human HGF, as well as a nucleic acid construct, a vector, a cell and a drug which comprise the nucleic acid, and the nucleic acid has a protein expression level superior to that of the natural sequence.

### BRIEF DESCRIPTION OF THE DRAWINGS

For more clearly illustrating the embodiments of the present invention or technical solutions in the prior art, accompanying drawings required to be used in embodiments or description of the prior art will be simply discussed below, obviously, the drawings in the description below are only some embodiments of the present invention, and other drawings can also be made by persons of ordinary skill in the art according to these drawings without creative efforts.

FIG. 1 is an agarose gel electrophoresis graph of in-vitro transcription product mRNA.

FIG. 2 shows an expression level of luciferase mRNA in HSMC cells after luciferase mRNA and HGF mRNA are co-transfected.

FIG. 3 shows an absolute expression level of HGF mRNA in HSMC cells after luciferase mRNA and HGF mRNA are co-transfected.

FIG. 4 shows a relative expression level of HGF mRNA in HSMC cells after luciferase mRNA and HGF mRNA are co-transfected.

### DETAILED DESCRIPTION OF THE EMBODIMENTS

To make the objectives, technical solutions and advantages of the present invention more clear, the technical solution of the present invention will be described in detail below. Obviously, the described embodiments are only a part of embodiments of the present invention, but not all the embodiments. Based on the embodiments of the present invention, other embodiments made by persons of ordinary skill in the art without creative efforts are all included within the protective scope of the present invention.

Reagents, raw materials or equipment used in the present invention, unless otherwise stated, are all commercially available.

In the present disclosure, an artificial intelligence algorithm is used to predict the structure stability, a series of nucleic acid molecules whose folding free energy is lower than that of the natural sequence are designed and actually synthesized, and their activities are determined. In some specific embodiments, the nucleic acid encoding human HGF comprises one or more open reading frames (ORFs), and the ORF nucleic acid sequence is at least 80%, at least 85%, at least 90%, at least 95%, at least 96%, at least 97%, at least 98%, at least 99% or 100% identical to a nucleic acid sequence of SEQ ID NOs: 21-31. The nucleic acid has a protein expression level superior to that of the natural sequence.

Preferably, the ORF nucleic acid sequence is selected from SEQ ID NOs: 21-31, or a transcribed RNA sequence thereof.

Preferably, the nucleic acid further comprises a 5′cap.

Preferably, the 5′cap is selected from m7G5′ppp5′Np, m7G5′ppp5′NmpNp and m7G5′ppp5′NmpNmpNp.

Preferably, the nucleic acid further comprises 5′UTR.

Preferably, the 5′UTR comprises one sequence of SEQ ID NOs: 1-8, or a combination thereof.

Preferably, the nucleic acid further comprises 3′UTR.

Preferably, the 3′UTR comprises one sequence of SEQ ID NOs: 9-17, or a combination thereof.

Preferably, the nucleic acid further comprises a poly-A region comprising 70-150 nucleotides in length.

Preferably, the poly-A region comprises the sequence set forth in SEQ ID NO: 18 or 19.

Preferably, the nucleic acid comprises sequences selected from SEQ ID NOs: 32 and 33, 34, 35, 56, 57, 58, 60, 61, 63 and 65, or a transcribed RNA sequence thereof.

Preferably, the nucleic acid comprises one or more modified nucleosides selected from pseudouridine, N1-methyl-pseudouridine and 5-methylcytidine.

Preferably, the nucleic acid is DNA or mRNA.

Preferably, the nucleic acid has a protein expression level that is at least 10%, at least 20%, at least 30%, at least 40% or at least 50% higher than that of the natural sequence.

Provided is a vector comprising the nucleic acid.

Provided is a cell comprising the nucleic acid.

Provided is a pharmaceutical composition comprising the nucleic acid.

Provided is a method for expressing a polypeptide in a mammal. By the method, the cell is in contact with the nucleic acid or the pharmaceutical composition.

Next, the technical solution of the present disclosure will be further described in detail through embodiments in combination with drawings. However, the selected embodiments are only for illustrating the present disclosure, but not limiting the scope of the present disclosure.

### EXAMPLE 1 PREPARATION OF PLASMIDS

ORF sequences SEQ ID NOs: 20-31 encoding identical human HGF natural protein were synthesized, wherein SEQ ID NO: 20 was a natural nucleic acid sequence (NM_000601.6), and others were artificially designed nucleic acid sequences. DNA plasmids containing the ORF sequences and flanked upstream and downstream regulatory sequences were constructed, amplified and extracted. Examples of 5′UTR, 3′UTR and polyA in the upstream and downstream regulatory sequences include but are not limited to sequences in Table 1 and Table 2. The DNA plasmids containing SEQ ID NOs: 32-35 and 46-65 respectively encode mRNA products HGF1-24, and their corresponding relationships are as shown in Table 3.

TABLE 3
Corresponding relationships between a construct and
the contained PRF sequence, 5′UTR and 3′UTR

| | Plasmid | | | |
|---|---|---|---|---|
| | construction | ORF sequence | 5′ UTR | 3′ UTR |
| Number | SEQ ID NO. | SEQ ID NO. | SEQ ID NO. | SEQ ID NO. |
| HGF-1 | 46 | 20 | 1 | 9 |
| HGF-2 | 47 | 20 | 1 | 10 |
| HGF-3 | 48 | 36 | 1 | 9 |
| HGF-4 | 49 | 36 | 1 | 10 |
| HGF-5 | 50 | 37 | 1 | 9 |
| HGF-6 | 51 | 38 | 1 | 9 |
| HGF-7 | 32 | 21 | 1 | 9 |
| HGF-8 | 52 | 39 | 1 | 10 |
| HGF-9 | 53 | 40 | 1 | 10 |
| HGF-10 | 33 | 22 | 1 | 10 |
| HGF-11 | 54 | 41 | 1 | 9 |
| HGF-12 | 55 | 42 | 1 | 9 |
| HGF-13 | 56 | 23 | 1 | 9 |
| HGF-14 | 57 | 24 | 1 | 9 |
| HGF-15 | 34 | 25 | 1 | 9 |
| HGF-16 | 58 | 26 | 1 | 9 |
| HGF-17 | 59 | 43 | 1 | 9 |
| HGF-18 | 60 | 27 | 1 | 9 |
| HGF-19 | 61 | 28 | 1 | 9 |
| HGF-20 | 62 | 44 | 1 | 9 |
| HGF-21 | 63 | 29 | 1 | 9 |
| HGF-22 | 64 | 45 | 1 | 9 |
| HGF-23 | 35 | 30 | 1 | 9 |
| HGF-24 | 65 | 31 | 1 | 9 |

Natural sequences and ORFs corresponding to constructs with better activity are listed below.

SEQ ID NO. 20:
ATGTGGGTGACCAAACTCCTGCCAGCCCTGCTGCTGCAGCATGTC
CTCCTCCATCTCCTCCTGCTCCCCATCGCCATCCCCTATGCAGA
GGGACAAAGGAAAAGAAGAAATACAATTCATGAATTCAAAAAATC
AGCAAAGACTACCCTAATCAAAATAGATCCAGCACTGAAGATAAA
AACCAAAAAAGTGAATACTGCAGACCAATGTGCTAATAGATGTAC
TAGGAATAAAGGACTTCCATTCACTTGCAAGGCTTTTGTTTTTGA
TAAACCAAGAAAACAATGCCTCTGGTTCCCCTTCAATAGGATGTC
AAGTGGAGTGAAAAAAGAATTTGGCCATGAATTTGACCTCTATGA
AAACAAAGACTACATTAGAAACTGCATCATTGGTAAAGGACGCAG
CTACAAGGGAACAGTATCTATCACTAAGAGTGGCATCAAATGTCA
GCCCTGGAGTTCCATGATACCACACGAACACAGCTTTTTGCCTTC
GAGCTATCGGGGTAAAGACCTACAGGAAAACTACTGTCGAAATCC
TCGAGGGGAAGAAGGGGGACCCTGGTGTTTCACAAGCAATCCAGA
GGTACGCTACGAAGTCTCTGACATTCCTCAGTGTTCAGAAGTTGA
ATGCATGACCTGCAATGGGGAGAGTTATCGAGGTCTCATGGATCA
TACAGAATCAGGCAAGATTTGTCAGCGCTGGGATCATCAGACACC
ACACCGGCACAAATTCTTGCCTGAAAGATATCCCGACAAGGGCTT
TGATGATAATTATTGCCGCAATCCCGATGGCCAGCCCAGGCCATG
GTGCTATACTCTTGACCCTCACACCCGCTGGGAGTACTGTGCAAT
TAAAACATGCGCTGACAATACTATGAATGACACTGATGTTCCTTT
GGAAACAACTGAATGCATCCAAGGTCAAGGAGAAGGCTACAGGGG
CACTGTCAATACCATTTGGAATGGAATTCCATGTCAGCGTTGGGA
TTCTCAGTATCCTCACGAGCATGACATGACTCCTGAAAATTTCAA
GTGCAAGGACCTACGAGAAAATTACTGCCGAAATCCAGATGGGTC
TGAATCACCCTGGTGTTTTACCACTGATCCAAACATCCGAGTTGG
CTACTGCTCCCAAATTCCAAACTGTGATATGTCACATGGACAAGA
TTGTTATCGTGGGAATGGCAAAAATTATATGGGCAACTTATCCCA
AACAAGATCTGGACTAACATGTTCAATGTGGGACAAGAACATGGA
AGACTTACATCGTCATATCTTCTGGGAACCAGATGCAAGTAAGCT
GAATGAGAATTACTGCCGAAATCCAGATGATGATGCTCATGGACC
CTGGTGCTACACGGGAAATCCACTCATTCCTTGGGATTATTGCCC
TATTTCTCGTTGTGAAGGTGATACCACACCTACAATAGTCAATTT
AGACCATCCCGTAATATCTTGTGCCAAAACGAAACAATTGCGAGT
TGTAAATGGGATTCCAACACGAACAAACATAGGATGGATGGTTAG
TTTGAGATACAGAAATAAACATATCTGCGGAGGATCATTGATAAA
GGAGAGTTGGGTTCTTACTGCACGACAGTGTTTCCCTTCTCGAGA
CTTGAAAGATTATGAAGCTTGGCTTGGAATTCATGATGTCCACGG
AAGAGGAGATGAGAAATGCAAACAGGTTCTCAATGTTTCCCAGCT
GGTATATGGCCCTGAAGGATCAGATCTGGTTTTAATGAAGCTTGC
CAGGCCTGCTGTCCTGGATGATTTTGTTAGTACGATTGATTTACC
TAATTATGGATGCACAATTCCTGAAAAGACCAGTTGCAGTGTTTA
TGGCTGGGGCTACACTGGATTGATCAACTATGATGGCCTATTACG
AGTGGCACATCTCTATATAATGGGAAATGAGAAATGCAGCCAGCA
TCATCGAGGGAAGGTGACTCTGAATGAGTCTGAAATATGTGCTGG
GGCTGAAAAGATTGGATCAGGACCATGTGAGGGGGATTATGGTGG
CCCACTTGTTTGTGAGCAACATAAAATGAGAATGGTTCTTGGTGT
CATTGTTCCTGGTCGTGGATGTGCCATTCCAAATCGTCCTGGTAT
TTTTGTCCGAGTAGCATATTATGCAAAATGGATACACAAAATTAT
TTTAACATATAAGGTACCACAGTCA
SEQ ID NO. 21:
ATGTGGGTAACAAAATTGCTACCTGCATTGCTGCTACAGCATGTT
CTGCTGCATCTGCTGCTCCTCCCCATAGCGATCCCCTATGCGGA
GGGGCAGCGGAAGCGGCGGAACACCATACATGAGTTTAAGAAGTC
TGCGAAGACTACTCTCATCAAAATCGATCCCGCCCTGAAGATAAA
GACGAAGAAGGTGAATACCGCAGACCAGTGCGCGAACCGTTGTAC
GAGGAACAAAGGCCTGCCGTTCACGTGCAAGGCCTTTGTCTTCGA
CAAGGCTCGCAAGCACTGTCTGTGGTTTCCCTTCAACTCTATGTC
TTCAGGGGTGAAGAAGGAATTCGGACACGAATTCGATCTTTACGA
AAACAAGGACTATATACGTAATTGTATAATTGGGAAGGGGOGCTC
OTATAAGGGAACAGTCTCGATCACGAAGTCTGGCATAAAGTGCCA
GCCGTGGTCGAGTATGATCCCTCACGAGCACTCCTTCCTGCCCTC
CTCCTACCGGGGGAAAGACCTGCAGGAAAATTACTGCAGGAACCC
CCGGGGGGAGGAGGGCGGTCCTTGGTGCTTCACCTCCAACCCTGA
GGTGCGTTACGAAGTTTGTGACATACCTCAGTGTTCGGAGGTGGA
GTGCATGACCTGTAACGGGGAGTCTTACCGGGGTCTGATGGACCA
CACCGAGTCTGGCAAAATTTGCCAGCGGTGGGACCATCAGACCCC
GCACCGGCATAAATTTTTACCTGAGCGGTACCCCGATAAGGGTTT
CGACGATAACTATTGTCGAAACCCTGACGGGCAGCCGCGTCCGTG
GTGTTACACGTTGGATCCGCACACGCGATGGGAGTACTGTGCAAT
CAAGACGTGTGCGGATAATACGATGAACGATACGGACGTGCCCCT
GGAGACCACGGAGTGTATCCAGGGGCAGGGTGAAGGGTATCGGGG
GACAGTGAACACCATCTGGAATGGGATCCCGTGTCAGCGCTGGGA
CTCTCAGTACCCGCATGAGCACGACATGACCCCGGAGAACTTTAA
ATGTAAAGATCTCCGGGAGAATTACTGTCGTAATCCTGACGGGTC
TGAGAGTCCCTGGTGCTTCACGACGGATCCCAACATCCGGGTGGG
TTACTGTTCCCAGATACCCAACTGTGACATGAGTCACGGTCAGGA
TTGTTATCGGGGTAACGGCAAGAATTACATGGGCAACTTGTCGCA
GACGCGCTCCGGTCTCACATGCAGCATGTGGGACAAGAATATGGA
GGATCTACATCGTCACATATTCTGGGAGCCGGATGCGACCAAGTT
GAACGAGAATTATTGCCGTAACCCCGATGACGATGCTCATGGTCC
CTGGTGTTATACGGGAAACCCGTTGATACCATGGGACTATTGCCC
GATTTCTCGTTGCGAGGGGGATACGACGCCCACGATCGTTAATCT
GGACCACCCCGTGATATCTTGTGCGAAGACGAAGCAGCTCCGTGT
AGTCAATGGGATTCCGACACOGACTAACATTGGCTGGATGGTGTC
GCTTCGTTATCGCAACAAGCATATCTGCGGGGGGTGCTTGATTAA
GGAATCGTGGGTGCTGACGGCCCGTCAGTGTTTCCCCAGCCGGGA
CCTCAAGGATTATGAGGCCTGGCTGGGGATACACGACGTACACGG
GCGGGGGGACGAGAAGTGCAAACAAGTACTTAACGTCTCCCAGCT
CGTGTACGGGCCGGAGGGATCAGATCTCGTTCTCATGAAATTAGG
GCGACCTGCTGTCCTCGATGATTTTGTGAGCACTATGGATCTCCC
TAATTACGGGTGCACCATCCCGGAGAAGACCAGTTGTAGTGTATA
TGGATGGGGCTACACTGGGCTCATCAACTACGACGGTCTTCTGGG
GGTGGCGCACCTGTATATTATGGGGAACGAGAAGTGCTCACAACA
TCATCGAGGCAAGGTCACGCTGAATGAGAGCGAGATCTGCGCAGG
GGCCGAGAAGATAGGTTCCGGCCCCTGCGAGGGGGACTACGGTGG
TCCCCTTGTATGTGAGCAGCATAAGATGAGGATGGTCTTGGGAGT
GATTGTCCCCGGCCGGGGGTGTGCAATACCCAACCGGCCGGGGAT
ATTCGTCCGCGTGGCGTACTACCGCAAGTGGATCCACAAGATCAT
CCTCACTTATAAGGTTCCACAGTCC
SEQ ID NO. 22:
ATGTGGGTGACAAAGTTGTTACCGGCCCTGTTACTTCAGCATGTC
TTGCTTCACTTGCTTCTGTTGCCGATCGCCATTCCTTACGCTGA
GGGTCAGGGTAAGCGCCGGAACACGATCCATGAATTCAAGAAGTC
TGCGAAGACTACCTTGATCAAGATCGATCCGGCGCTAAAAATCAA
GACTAAGAAGGTGAATACCGCAGACCAGTGCGCGAACCGTTGTAC
GAGGAACAAGGGGCTGCCGTTCACGTGCAAGGCCTTTGTCTTCGA
CAAGGCTCGCAAGCAGTGTCTGTGGTTTCCCTTCAATAGTATGAG
CTCGGGGGTCAAGAAGGAGTTCGGGCATGAGTTCGATCTCTACGA
GAACAAGGACTACATCCGGAACTGCATCATTGGGAAGGGGCGCTC
GTACAAGGGGACCGTGTCGATCACGAAGTCTGGCATCAAGTGCCA
GCCGTGGTCGAGCATGATCCCTCACGAGCACTCCTTCTTGCCCTC
GAGCTACCGCGGGAAGGACCTGCAGGAGAATTACTGCAGGAACCC
CCGCGGTGAGGAGGGCGGTCCATGGTGCTTCACCAGCAATCCGGA
GGTCAGGTACGAGGTATGCGACATACCTCAGTGCTCTGAGGTGGA
GTGCATGACATGTAACGGCGAGAGTTACAGGGGCCTGATGGATCA
TACCGAATCGGGCAAGATTTGTCAGCGGTGGGATCACCAGACTCC
GCACCGCCACAAGTTCTTGCCGGAGCGGTATCCGGATAAGGGTTT
CGACGATAACTATTGTCGAAACCCTGACGGTCAGCCTCGTCCATG
GTGCTACACGCTTGATCCGCATACCGGGTGGGAGTACTGCGCGAT
CAAGACGTGTGCCGATAACACCATGAACGACACTGACGTCCCATT
GGAGACAACCGAGTGCATACAGGGACAGGGCGAGGGTTATCGGGG
TACTGTGAACACCATCTGGAACGGGATGCCGTGCCAGAGGTGGGA
TTCACAGTACCCCCACGAGCATGACATGACTCCTGAGAATTTCAA
GTGCAAGGATCTCCGAGAGAACTACTGTGGGAACCGGGACGGTTC
GGAGTCGCCGTGGTGCTTCACCACGGACCCGAACATCCGGGTGGG
GTACTGTTCGCAGATCCCCAACTGCGACATGTCTCACGGGCAGGA
CTGTTACCGGGGGAACGGGAAGAACTATATGGGCAACCTGTCGCA
GACACGGAGCGGGCTAACCTGCTGCATGTGGGATAAGAATATGGA
GGACCTCCATAGGCATATCTTCTGGGAGCCGGATGCCTCTAAGCT
CAATGAGAATTATTGCCGGAACCCGGATGATGATGCTCATGGGCC
GTGGTGTTATACGGGGAACCCGTTGATACCGTGGGACTACTGTCC
CATCTCGCGATGCGAGGGGGACACTACTCCCACTATAGTTAACCT
GGACCACCCGGTGATATCCTGCGCGAAGACAAAGCAGTTGCGGGT
CGTGAACGGTATCCCCACCCGGACCAACATAGGTTGGATGGTCAG
CCTCCGCTACAGAAATAAGCATATCTGTGGCGGGTCGCTGATCAA
GGAGTCATGGGTTCTGACAGCCAGGCAGTGGTTTCCGTCCCCCGA
CTTGAAGGACTACGAGGCCTGGTTGGGCATTCATGACGTACACGG
GCGGGGAGACGAGAAGTGCAAGCAAGTACTTAACGTCTCCCAGCT
CGTGTACGGTCCTGAAGGGTCCGACCTGGTCTTGATGAAGCTGGC
CGGCCCTGCTGTATTGGATGACTTCGTGTCAACCATAGATTTGCC
GAACTATGGTTGCACGATCCCTGAGAAGACTAGTTGCTCGGTGTA
CGGGTGGGGCTACACAGGGCTCATCAACTATGATGGGCTGCTGGG
CGTAGCCCACCTGTACATCATGGGCAACGAGAAGTGTAGCCAGCA
TCACCGGGGCAAGGTCACTCTCAACGAGAGTGAGATTTGCGCCGG
TGCCGAGAAAATCGGCAGCGGCCCCTGGGAGGGCGATTACGGAGG
GCCGCTCGTCTGTGAGCAGCACAAGATGAGGATGGTCCTCGGGGT
GATCGTGCCTGGCAGGGGCTGCGCGATACCTAATCGTCCGGGGAT
GTTCGTCCGCGTGGCGTACTACGCCAAGTGGATACATAAGATCAT
CCTGACGTATAAGGTACCGCAGAGC
SEQ ID NO. 23:
ATGTGGGTAACCAAGTTGCTTCCCGCTCTTCTTCTGCAGCATGTA
CTGTTGCATCTGCTTCTGCTGCCCATCGCTATCCCGTACGCGGA
GGGGCAGCGGAAGCGGCGCAACACCATACATGAGTTCAAGAAGAG
TGCGAAGACAACTCTTATCAAGATGGACCCCCCCCTGAAGATAAA
GACGAAGAAGGTGAACACTGCAGACCACTGTGCGAACCGCTGCAC
GAGGAACAAAGGTCTTCCATTCACATGCAAGGCCTTTGTTTTCGA
CAAGGCTCGCAAGCAGTGTCTGTGGTTTCCCTTCAACTCTATGTC
TTCAGGTGTGAAGAAGGAATTGGGCCACGAATTGGATGTTTACGA
CAACAAGGACTACATCCGTAATTGTATTATGGGGAAGGGCAGGTC
TTACAAGGGCACAGTCTCCATAACGAAGTCGGGGATCAAGTGTCA
GCCATGGTCGAGTATGATTCCTCATGAGCACAGTTTCCTTCCTTC
TTCCTACCGGGGGAAGGACCTGCAGGAAAACTACTGTGGCAACCC
CCGAGGGGAGGAGGGAGGACCTTGGTGCTTCACTTCGAACCCCGA
GGTGAGGTACGAGGTATGCGACATACCTCAGTGCTCTGAGGTAGA
GTGCATGACGTGCAATGGGGAATCATACAGGGGTCTGATGGATCA
CACCGAGTCTGGCAAGATTTGTCAACGGTGGGATCATCAGACTCC
TCATCGGCACAAGTTCCTTCCCGAACGGTACCCCGACAAGGGTTT
CGACGATAACTATTGTCGAAACCCTGACGGGCAGCCTCGACCATG
GTGCTACACACTTGATCCCCACACTGGTTGGGAGTACTGTGCCAT
TAAGACCTGGGCCGATAATACAATGAACGACACGGATGTGCCCCT
TGAGACGACTGAGTGCATCCAGGGTCAGGGTGAAGGGTATCGGGG
CACCGTGAATACCATCTGGAATGGGATCCCTTGTCAGGGCTGGGA
CTCTCAGTACCCTCACGAGCACGACATGACTCCAGAGAACTTTAA
GTGTAAGGATCTCGGGGAGAACTACTGTCGTAATCCTGATGGTTC
TGAGAGTCCCTGGTGCTTCACAACGGATCGCAATATCAGGGTTGG
GTACTGCAGCCAGATACCTAACTGTGATATGAGCCATGGGCAGGA
CTGTTATCGGGGTAACGGCAAAAATTACATGGGCAACTTGTCGCA
GACGCGCTCCGGGCTGACGTGCTCGATGTGGGACAAAAACATGGA
GGATCTACATCGCCACATCTTCTGGGAGCCCGATGCGTCCAAGTT
GAATGAGAATTATTGCCGTAACCCTGATGACGACGCTCATGGTCC
CTGGTGTTACACAGGTAACCCCCTGATACCCTGGGACTACTGTCC
TATCTGGCGTTGTGAGGGTGACACTACCCCTACGATTGTGAATCT
TGATCACCCTGTGATATCGTGTGCCAAAACGAAGCAACTACGCGT
AGTTAATGGGATCCCTACCGGCACCAATATAGGCTGGATGGTGTC
TCTCAGGTATGGGAACAAGCATATTTGCGGTGGGTCGCTGATAAA
GGAGTCGTGGGTCCTCACGGCCAGGCAGTGTTTTCCTTCGAGGGA
CTTGAAGGACTACGAGGCCTGGCTGGGGATCCACGACGTCCACGG
ACGGGGGGACGAGAAGTGCAACCAGGTCCTGAATGTGAGTCAGCT
GGTCTACGGACCAGAGGGTTCGGATTTAGTCCTCATGAAGCTGGC
TCGTCCTGCCGTTCTGGATGACTTCGTGTCCACGATCGACCTACC
AAACTATGGTTGTACGATACCTGAGAAGACATCATGCAGCGTGTA
TGGGGGGGGTACACGGGTCTCATTAACTACGACGGGTTGCTTCGT
GTGGCACATCTCTATATCATGGGCAATGAAAAGTGTTCACAGCAC
CATAGGGGTAAGGTCACTCTCAACGAGAGGGAGATATGTGCTGGC
GCGGAGAAGATTGGCTCCGGTCCCTGCGAGGGAGACTACGGTGGC
CCCCTGGTCTGTGAGCAGCACAAGATGCGCATGGTCCTGGGGGTC
ATAGTCCCTGGCAGGGGCTGTGCCATTCCCAACCGTCCCGGCATA
TTTGTTGGCGTGGCGTACTACGCCAAGTGGATACACAAGATCATT
CTGACCTACAAGGTGGCTCAGTCG
SEQ ID NO. 24:
ATGTGGGTCACGAAGTTGGTTCCCGCTCTTCTTCTGCAGCATGTG
GTGTTGCACCTGCTTCTGCTGCCCATCGCTATCCCGTACGCGGA
GGGGCAGCGGAAGCGGCGCAACACGATACATGAGTTCAAGAAGAG
CGCGAAGACGACTCTGATCAAGATCGATCCGGCCCTGAAGATAAA
GACGAAGAAGGTGAACACCGGGGACCAGTGTGCGAACCGTTGTAC
GAGGAACAAGGGCCTTCCATTCACGTGCAAGGCCTTTGTCTTCGA
CAAGGCTCGCAAGCAGTGTCTGTGGTTTCCCTTCAACTCTATGTC
TTCGGGCGTGAAGAAGGAGTTCGGGCACGAGTTCGATCTTTACGA
GAACAAGGACTACATCCGTAATTGTATTATCGGGAAGGGCAGGTC
GTATAAGGGCACGGTGTCTATCACCAAGTCGGGGATCAAGTGTCA
GCCCTGGTCGAGTATGATTCCCCATGAGCACAGTTTCCTGCCCTC
CTCCTACCGGGGGAAGGACCTGCAGGAGAACTACTGCAGGAACCC
CCGGGGGGAGGAGGGGGGGCCTTGGTGCTTCACCTGGAACCCCGA
GGTGAGGTACGAGGTGTGCGACATCCCTCAGTGCTCTGAGGTGGA
GTGCATGACGTGCAATGGGGAGTCGTACCGGGGTCTGATGGACCA
CACCGAGAGTGGCAAGATTTGCCAGCGGTGGGACCATCAGACCGC
TCATAGGCACAAGTTTCTTCCTGAGAGGTACCCGGACAAGGGTTT
CGACGATAACTATTGTCGGAACCCGGACGGGCAGCCTCGGCCGTG
GTGCTACACGCTTGATCCCCACACTCGGTGGGAGTACTGTGCCAT
TAAGACCTGCGCCGATAATACGATGAACGACACGGATGTGGGGCT
GGAGACGACTGAGTGCATCCAGGGTCAGGGTGAGGGGTATCGGGG
GACCGTGAACACCATCTGGAATGGGATCCCGTGCCAGCGCTGGGA
CTCTCAGTACCCCCACGAGCACGACATGACTCCCGAGAACTTTAA
GTGTAAGGATCTGCGGGAGAATTACTGTCGTAATCCTGACGGGTC
TGAGAGTCCCTGGTGCTTCACGACGGATCCCAATATCAGGGTTGG
GTACTGCAGCCAGATACCCAACTGTGATATGAGCCATGGGCAGGA
TTGTTATCGGGGTAACGGCAAGAATTACATGGGCAACTTGTCGCA
GACGGGCTCCGGGCTGACGTGCTCGATGTGGGATAAGAACATGGA
GGACTTGCATCGGCACATCTTCTGGGAGCCCGACGCGAGCAAGTT
GAACGAGAATTATTGCGGTAACCCCGATGACGACGCCCATGGCCC
GTGGTGTTACACGGGGAACCCGCTGATACCCTGGGACTACTGCCC
GATCTCGCGCTGTGAGGGTGACACTACCCCTACGATCGTGAACCT
TGATCACCCCGTGATATCGTGCGCCAAGACGAAGCAGCTGCGCGT
GGTTAATGGGATCGCTACCCGCACCAATATAGGCTGGATGGTGTC
TCTCAGGTATCGTAACAAGCATATTTCCGGCGGCTCCCTGATAAA
GGAGTCGTGGGTCCTGACGGCCAGGCAGTGTTTTCCTTCGAGGGA
CTTGAAGGACTACGAGGCCTGGCTGGGGATCCACGACGTCCACGG
GCGGGGGGACGAGAAGTGCAAGCAGGTGCTGAATGTGAGCCAGCT
GGTCTACGGGCCGGAGGGCTCGGATTTGGTCCTCATGAAGGTTGC
TCGTCCCGCCGTCCTGGACGACTTTGTCACCACGATCGACCTGCC
GAACTATGGTTGTACGATACCTGAGAAGACGTCGTGCAGCGTGTA
TGGGTGGGGGTACACGGGTCTCATTAACTACGACGGGCTGCTTCG
CGTGGCGCACCTGTATATCATGGGGAATGAGAAGTGTTCGCAGCA
TCATAGGGGGAAGGTCACGCTCAACGAGAGGGAGATCTGTGCCGG
GGCGGAGAAGATTGGCAGCGGCCCCTGCGAGGGGGACTACGGTGG
CCCGCTGGTATGCGAGCAGCATAAGATGCGCATGGTGCTGGGGGT
CATCGTGCCCGGCAGGGGCTGCGCCATTCCGAACCGCCCCGGCAT
CTTTGTCCGCGTGGCGTACTACGCCAAGTGGATACACAAGATCAT
CCTGACCTACAAGGTGCCTCAGTCG
SEQ ID NO. 25:
ATGTGGGTGACCAAGCTCCTGCCGGCCCTGCTGCTCCAGCATGTC
CTTCTGCATCTCCTCCTGCTACCCATAGCGATCCCCTATGCAGA
AGGACAAAGGAAACGCAGAAATACAATTCATGAATTCAAGAAATC
AGCGAAGACTACCCTAATCAAGATAGATCCAGCACTGAAGATAAA
AAACCAAGAAAGTGAATACCGCAGACCAGTGTGCTAACCGTTGTA
CGAGGAACAAAGGACTGCCATTCACTTGCAAGGCTTTTGTTTTTG
ATAAGGCGCGTAAACAATGCTTGTGGTTCCCCTTCAACAGCATGT
CAAGTGGCGTGAAGAAAGAGTTCGGACACGAATTTGACCTCTATG
AGAACAAAGACTACATTAGGAACTGCATCATGGGTAAGGGACGCT
CGTATAAGGGCACAGTTTCTATCACTAAGAGTGGCATTAAATGTC
AGCCCTGGTCGTCCATGATACCACACGAACACAGCTTTCTTCCTT
CTTCCTATCGCGGAAAGGACTTGCAGGAAAACTACTGTCGCAATC
CGCGAGGGGAAGAAGGGGGACCCTGGTGCTTCACAAGCAACCGTG
AGGTACGCTACGAAGTCTGTGACATTCCTCAGTGTTCCGAAGTTG
AATGCATGACCTGCAACGGGGAGTCGTATCGAGGTCTCATGGATC
ACACAGAATCAGGCAAGATTTGTCAGCGGTGGGATCACCAGACCC
CCCATCGCCACAAATTCTTGCCTGAACGATACCCTGACAAGGGTT
TTGATGATAATTACTGCCGTAACCCCGACGGCCAGCCGAGGCCCT
GGTGTTACACTCTTGACCCTCACACCCGATGGGAGTACTGTGCTA
TTAAGACGTGGGGGGACAATACTATGAATGACACTGATGTGCCTT
TGGAGACAACTGAGTGCATACAAGGTCAAGGCGAAGGCTACCGGG
GTACTGTGAATACAATTTGGAATGGGATACCATGTCAGAGATGGG
ATTCGCAGTACCCTCACGAGCATGACATGACTCCTGAAAATTTCA
AGTGCAAGGACCTACGAGAGAATTACTGCCGAAATCCAGATGGGT
CTGAGAGCCCCTGGTGCTTTACCACTGATCCGAACATCAGAGTTG
GTTACTGCTGCCAAATACCAAACTGTGATATGTCGCACGGACAAG
ATTGCTATCGGGGGAATGGCAAAAACTACATGGGCAACCTGAGTC
AAACAAGATCTGGACTAACATGTTCTATGTGGGACAAGAACATGG
AAGATCTTCATCGTCATATCTTCTGGGAACCGGATGCAAGTAACC
TGAACGAGAATTACTGCAGAAATCCAGACGATGATGCTCACGGAC
CCTGCTGCTACACGGGAAATCCACTCATTGCTTGGGATTACTGCC
CCATTTCTGGTTGTGAAGGTGACACCACACCTACCATAGTCAACC
TGGACCATGCCGTTATATCATGTGCCAAAACGAAACAATTGCGAG
TTGTCAATGGGATGCCAACTGGAACTAACATGGGATGGATGGTTT
CCCTCAGATACCGTAACAAACATATCTGCGGGGGATCATTGATCA
AGGAGAGTTGGGTTCTTACGGCAAGGCAGTGTTTGCGTTGGCGAG
ACTTGAAGGATTACGAAGCTTGGCTTGGAATTCACGATGTCCACG
GAAGAGGAGATGAGAAATGCAAACAGGTTCTCAATGTTTCGCAGC
TTGTATATGGCCCGGAAGGATCAGATCTGGTGTTAATGAAGTTAG
CCAGGCGGGCCGTCCTGGATGATTTCGTTAGTACAATCGATCTTG
CCAATTATGGTTGCACAATCCCGGAGAAGACCAGTTGTAGCGTCT
ATGGCTGGGGCTACACTGGATTGATCAACTATGATGGGCTATTAC
GACTGGCACATCTCTATATAATGGGAAATGAGAAATGCTCGCAGC
ATCACCGAGGGAAGGTGACTCTGAACGAGTCGGAAATATGTGCTG
GGGCCGAGAAGATTGGTTCTGGCCCATGTGAGGGGGATTACGGTG
GCCCACTGGTTTGTGAGCAACACAAAATGAGGATGGTTCTTGGTG
TTATTGTTCCTGGTCGGGGATGTGCCATTCCAAACCGTCCTGGTA
TTTTTGTCCGTGTGGCATATTACGCAAAATGGATACACAAGATTA
TTCTCACCTATAAGGTACCCCAGTCA
SEQ ID NO. 26:
ATGTGGGTGACCAAACTGCTGCCGGCCCTGCTGCTGCAGCATGTC
CTCCTGCATCTCCTCCTGCTGCCCATCGCCATACCCTATGCCGA
GGGGCAGAGGAAGAGACGTAACACGATTCATGAATTCAAAAAATC
AGCAAAGACTACTCTTATCAAGATTGATCCAGCACTGAAGATAAA
GACCAAGAAGGTGAACACGGGGGACCAGTGTGCCAATAGGTGTAC
TAGGAATAAGGGTCTGCCGTTCACTTGCAAGGCGTTTGTTTTTGA
CAAAGCGAGAAAACAATGCCTCTGGTTCGCTTTCAATAGCATGTC
AAGCGGGGTTAAGAAGGAATTTGGCCACGAATTTGACCTCTATGA
AAACAAGGACTACATTGGTAACTGTATCATTGGTAAAGGACGAAG
CTACAAGGGAACAGTCTCTATCACTAAGAGTGGCATCAAGTGTCA
GCCCTGGAGTTCGATGATACCTCACGAGCACAGCTTTCTGCCTTC
GAGCTATGGGGGTAAGGATCTTCAGGAGAACTACTGTCGAAATCC
TCGGGGGGAAGAGGGGGGCCCCTGGTGTTTCACAAGCAATCCAGA
GGTGGGCTACGAAGTCTGTGACATTCCGCAGTGTTCAGAGGTTGA
GTGCATGACATGCAATGGGGAGAGTTATCGGGGTCTCATGGATCA
TACCGAGTCAGGCAAGATTTGTCAGCGGTGGGATCATCAGAGGCC
GCACCGCCACAAATTCTTGCCTGAGGGGTATCCCCACAAGGGCTT
TGATGATAATTATTGCCGGAATCCGGATGGCCAGCCGAGGCCGTG
GTGCTATACTTTAGACCCTCACACCCCCTGGGAGTACTGTGCAAT
TAAGACATGCGCTGACAATACGATGAATGACACTGATGTTCCTTT
GGAGACAACGGAATGTATCCAAGGTCAGGGAGAAGGCTACAGGGG
CACTGTCAATACCATTTGGAATGGAATTCCATGTCAGCGGTGGGA
TTCGCAGTATCCTCACGAGCATGACATGACGCCTGAAAATTTCAA
GTGCAAGGACTTGCGAGAGAACTACTGCCGAAATCCAGACGGGTC
GGAATCACCGTGGTGTTTTACCACTGATCCGAACATCCGAGTTGG
GTACTGCTCCCAAATTCCAAACTGCGATATGTCCCATGGGCAGGA
TTGTTATCGTGGGAATGGCAAGAATTATATGGGCAATCTGTCCCA
GACAAGAAGCGGATTGACGTGTAGTATGTGGCACAAGAACATGGA
GGACCTCCATCGTCATATCTTCTGGGAGCCAGATGCCAGTAAGCT
TCAATGAGAATTACTGTCGGAATCCGGACGATGATGCTCATGGAC
CCTGGTGCTACACGGGCAATCCGCTCATTCCTTGGGATTATTGCC
CTATTTCTCGTTGTGAGGGTGATACCACGGCTACAATACAATTTA
GATCATGCCGTAATATCATGTGCCAAAACGAAACAATTGCGAGTT
GTAAATGGGATTCCGACGGGCACAAACATAGGGTGGATGGTTAGT
TTGAGATACCGTAATAAGCATATCTGCGGAGGATCATTGATCAAG
GAGAGTTGGGTTCTGACAGCTCGGCAGTGTTTCCCTTCTGGGGAC
CTTAAGGATTATGAGGCTTGGCTTGGGATTCATGATGTCCACGGG
AGGGGAGACGAGAAATGCAAGCAGGTTCTCAATGTTTGGCAGTTG
GTGTATGGTCCTGAGGGATCGGATCTGGTGCTGATGAAGTTGGCC
AGGCCTGCGGTCCTGGACGATTTTGTTAGTACGATTGATTTGCCT
AATTATGGTTGCACAATTCCGGAGAAGACCAGTTGCAGTGTGTAT
GGGTGGGGCTACACTGGGTTGATCAACTATGATGGGCTACTGCGA
GTGGCACATTTGTACATAATGGGGAATGAGAAGTGCTCCCAGCAT
CATCGGGGGAAGGTGACTCTGAATGAGTGGGAAATATGTGCTGGG
GCTGAGAAGATTGGATCAGGACCGTGTGAGGGGGATTATGGTGGG
CCATTGGTATGTGAGCAGCATAAGATGAGGATGGTTTTGGGTGTC
ATCGTTCCGGGTGGTGGATGTGCTATTCCGAATAGGCCCGGTATA
TTTGTCCGAGTAGCATACTACGGGAAATGGATACACAAAATTATA
CTCACATATAAGGTTCCACAGTCA
SEQ ID NO. 27:
ATGTGGGTGACAAAGCTGTTGCCTGCGTTATTGCTGCAGCACGTT
CTGCTGCATCTGCTGCTCCTGCCCATAGCGATACCCTATGCGGA
GGGGCAGCGGAAGCGGCGGAACACTATTCATGAGTTTAAGAAGAG
GGCCAAGACGACTTTGATAAAGATCGATCCGGCGCTCAAGATTAA
AACCAAGAAGGTGAATACCGCAGATCAGTGCGCCAACAGGTGCAC
TCGAAACAAGGGCCTGCCGTTCACGTGCAAGGCCTTTGTTTTCGA
CAAGGCTCGAAAGCAGTGCCTGTGGTTCCCTTTTAACTCCATGAG
CAGTGGAGTTAAGAAGGAATTCGGGCACGAGTTCGACCTGTACGA
GAATAAAGATTACATACGTAATTGTATTATTGGTAAGGGGAGATC
GTACAAGGGTACGGTCTCCATTACCAAGTCGGGCATCAAGTGCCA
ACCGTGGAGCTCGATCATACCTCACGAGCATTCATTCCTGGGCTC
CTCCTACGGGGGGAAGGACCTGCAGGAGAATTACTGCAGGAACCC
CCGGGGGGAGGAGGGCGGGCCGTGGTGCTTCACGAGCAACCCGGA
GGTTCGGTACGAGGTGTGCGACATCCCGCAGTGTACCGAGGTCGA
GTGTATGACATGTAATGGCGAGTCTTACCGGGGTCTGATGGACCA
TACAGAGTCCGGTAAGATTTGCCAGCGGTGGGATCATCAGACCCC
TCATCGGCATAAGTTCCTGCCGGAGCGTTATCCCGATAAGGGGTT
TGATGATAACTACTGCCGCAACCGGGACGGGCAGCCCCGTCCGTG
GTGCTATACGCTGGACCCGCACACGCGGTGGGAGTACTGCGCTAT
TAAGACGTGCGCAGACAATACGATGÅATGACACTGACGTGCCCCT
GGAGACCACGGAGTGTATTCAGGGGCAGGGCGAGGGGTATCGGGG
AACGGTGAACACCATCTGGAATGGGATCCCGTGTCAGGGCTGGGA
CTCTCAGTACCCGCATGAGCACGACATGACCCCGGAGAACTTTAA
GTGTAAGGATCTCCGGGAGAATTATTGTCGTAATCCTGACGGGTC
TGAGAGTCCCTGGTGGTTCACGACGGATCCCAATATCCGGGTGGG
TTACTGTTCGCAGATACCCAATTGCGACATGTGGCATGGTCAGGA
CTGCTACCGGGGGAATGGGAAGAATTACATGGGTAATTTATCCCA
GACCCGGAGCGGCCTGACCTGCAGCATGTGGGATAAGAATATGGA
GGACCTCCATAGGCATATCTTTTGGGAGCCTGACGCCAGTAAGCT
CAATGAGAATTATTGTCGGAACCCCGACGATGATGCTCATGGGCC
CTGGTGTTATACGGGTAACCCCCTCATACCGTGGGATTATTGTCC
GATCTCGCGGTGTGAGGGGGATACCACGCCGACGATTGTCAACCT
TGACCATCCTGTCATTTCGTGTGCGAAGACAAAGCAGCTGCGGGT
GGTTAACGGGATTCCGACCCGAACCAACATTGGGTGGATGGTCTC
TCTCCGGTATCGTAACAAGCATATTTGTGGCGGGTCGCTCATAAA
GGAGAGTTGGGTCCTGACGGCCCGTCAGTGTTTCCCCAGCCGGGA
CCTCAAGGATTATGAGGCCTGGCTGGGGATACACGACGTGCACGG
GCGGGGGGACGAGAAGTGCAAGCAGGTGCTTAACGTCTCCCAGCT
CGTGTACGGGCCGGAGGGATCCGACTTGGTGCTGATGAAGTTGGC
CAGGCCAGCGGTCCTGGACGACTTCGTCAGCACCATCGACCTGCC
CAATTATGGTTGTACGATACCGGAGAAGACTTCTTGTTCCGTGTA
CGGGTGGGGCTACACGGGGCTCATCAACTATGATGGGCTCCTACG
TGTGGCCCACCTGTACATTATGGGGAACGAGAAGTGTTCTCAGCA
CCATCGCGGAAAGGTCACTCTCAATGAGAGTGAGATCTGGGCTGG
TGCTGAGAAGATTGGCAGCGGCCCCTGCGAGGGCGATTACGGAGG
GGCCCTCGTCTGTGAACAGCACAAGATGAGGATGGTCCTGGGCGT
GATCGTCCCTGGCAGGGGCTGCGCCATCCCGAATCGCCCGGGTAT
TTTCGTCCGCGTGGCGTACTACGCCAAGTGGATACACAAGATTAT
GTTGACGTATAAGGTTCCACAGTCA
SEQ ID NO. 28:
ATGTGGGTAACGAAACTGTTACCAGCACTACTGCTTCAGCATGTGC
TTCTGCATTTGTTATTGCTACCAATAGCAATACCATATGGGGAAGG
ACAGGGCAAACCTAGGAACACTATACACGAGTTCAAGAAATCCGGG
AAGACCACTCTCATAAAGATGGATCCCGCATTGAAGATAAAGACA
AAGAAGGTCAACAGGGCCGATCAGTGTGCCAACAGGTGTACGGGC
AACAAAGGGTTGCCGTTCACCTGTAAGGCTTTTGTCTTCGATAAG
GCGCGGAAGCAGTGCCTGTGGTTCCCTTTTAACTCCATGTCGAGT
GGAGTTAAGAAGGAATTCGGGCACGAGTTCGATGTTTATGAGAAC
AAGGATTATATAAGAAATTGTATAATCGGGAAGGGGCGCTGGTAC
AAGGGAACCGTCTCGATCACCAAGTCCGGCATCAAGTGCCAGCCG
TGGTCGTCGATGATCCCTCACGAGCACTCCTTCCTGCCCTCCTGC
TACCGCGGGAAGGACCTGCAGGAAAACTACTGCAGGAACCCCCGC
GGCGAGGAGGGGGGCCCTTGGTGCTTCACCTCCAACCGGGAGGTG
AGGTACGAAGTCTGTGACATCCCACAGTGTTCTGAGGTCGAGTGT
ATGACTTGCAACGGCGAGTCATACCGGCGGCCTCATGGATCACAC
GGAGTCAGGCAAGATTTGTCAGCGGTGGGATCACCAGACCCCCCA
CCGTCACAAGTTTTTGCCAGAGCGGTACCCCGACAAGGGTTTCGA
CGATAACTATTGTCGAAACCCTGACGGGCAGCCCCGACCGTGGTG
CTATACTCTCGACCCGCACACGAGATGGGAGTATTGCGCCATCAA
AACCTGTGCTGACAATACCATGAATGACACTGACGTGCCCTTGGA
AACCACGGAGTGCATCCAAGGGCAGGGTGAGGGTTATCGTGGTAC
TGTCAACACGATTTGGAATGGGATCCCTTGTCAGCGCTGGGACTC
TCAGTACCCTCATGAGCACGATATGACCCCGGAGAACTTTAAATG
TAAAGATCTCCGGGAAAATTACTGTCGTAATCCTGACGGTTCTGA
GAGTCCCTGGTGCTTCACGACGGATCCCAACATCCGGGTCGGGTA
CTGTTCTCAGATTCCCAACTGTGATATGTCCCATGGCCAGGATTG
CTATCGGGGCAATGGGAAAAATTACATGGGGAATCTGAGTCAGAC
CCGATCCGGACTGACTTGTTCCATGTGGGACAAGAATATGGAGGA
CCTGCACCGCCACATCTTTTGGGAGCCGGACGCTTCCAAACTCAA
TGAGAATTATTGCCGGAACCCGGACGATGATGCTCATGGCCCCTG
GTGCTACACCGGGAATCCATTGATACCGTGGGATTACTGTGCCAT
CTCGCGCTGCGAGGGGGACACTACTCCCACGATTGTTAATCTGGA
CCACCCTGTTATTAGTTGCGCTAAAACTAAACAATTACGTGTAGT
GAATGGGATACCCACTGGCACCAACATTGGTTGGATGGTTTCCCT
CCGGTACCGTAACAAGCACATTTGCGGTGGGAGCCTGATTAAGGA
GTCTTGGGTCCTGACCGCCAGGCAGTGTTTTGCTTCAAGGGACTT
GAAGGACTACGAGGCCTGGCTGGGCATCCACGATGTCCATGGTCG
GGGCGACGAGAAATGTAAACAGGTTCTCAACGTAAGTCAGCTTGT
CTACGGACCGGAGGGATCAGATCTCGTTCTCATGAAACTAGCGGG
ACCTGCGGTCCTCGATGATTTTGTGAGCACTATCGATCTCCCTAA
TTACGGGTGCACCATCCCGGAGAAGACCAGTTGTAGTGTCTACGG
ATGGGGCTACACTGGCCTCATTAACTACGACGGTCTTCTCCGTGT
GGCGCACCTGTACATTATGGGGAACGAGAAGTGCTCACAACATCA
TCGAGGAAAGGTCACGCTGAATGAGAGCGAGATCTGTGCAGGAGC
CGAGAAAATCGGCAGTGGGCCCTGCGAGGGCGACTATGGTGGTCC
GCTCGTGTGCGAACAGCACAAGATGGGGATGGTCCTGGGCGTCAT
TGTCCCCGGCCGAGGGTGTGCAATACCCAACCGGCCGGGGATATT
CGTCCGGGTGGCATATTATGCGAAGTGGATCCACAAGATCATACT
TACGTATAAAGTGCCACAATCC
SEQ ID NO. 29:
ATGTGGGTTACTAAGCTCCTGCCAGCACTGCTGCTCCAGCACGTC
CTCCTCCATCTGTTGCTCCTCCCCATAGCGATCCCCTATGGGGA
GGGGCAACGGAAGCGCCGGAACACGATCCATGAATTCAAGAAGTC
TGCAAAGACTACTTTAATCAAGATCGATCCGGCGCTCAAGATTAA
GACAAAGAAGGTCAACACGGCCGATCAGTGTGCTAATCGTTGCAC
CAGAAACAAAGGCCTGCCGTTCACGTGCAAGGCCTTTGTTTTTGA
CAAGGCCAGGAAGCAGTGCCTGTGGTTCCCTTTTAACTCCATGTC
CAGTGGAGTTAAGAAGGAATTGGGGCACGAATTTGATCTTTATGA
AAATAAAGATTACATTCGTAACTGCATCATTGGCAAGGGGCGCTC
GTACAAGGGAACGGTGAGCATCACCAAGTCGGGGATCAAGTGCCA
GCCCTGGTGCTCGATGATTCCTCACGAGCACTCCTTGCTGCCTTC
TTCCTACCGCGGGAAGGATCTCCAGGAGAATTACTGCCGTAATCC
TGGTGGAGAAGAGGGCGGGCCTTGGTGCTTCACTTGCAACCCTGA
GGTACGTTACGAGGTTTGTGACATACCTCAGTGTTCGGAGGTGGA
GTGCATGACTTGCAACGGCGAGTCATACCGGGGTCTGATGGACCA
CACCGAGTCTGGCAAGATTTGCCAACGGTGGGACCATCAGACTCC
GCATCGGCACAAGTTCTTGCCGGAGCGGTACCGCGACAAGGGTTT
CGACGATAACTATTGTCGAAACCCTGACGGGCAGCCGCGACCATG
GTGCTATACTCTCGACCCGCACACGCGTTGGGAGTATTGCGCCAT
CAAAACTTGTGCTGATAACACCATGAATGACACCGACGTCCGGCT
CGAGACGACGGAGTGCATTCAAGGTCAGGGGGAGGGGTATCGCGG
CACCGTCAATACCATCTGGAACGGGATACCGTGCCAGAGATGGGA
CAGCCAGTACCCCCACGAGCATGATATGACTGCGGAGAACTTCAA
GTGCAAGGATTTGCGTGAGAATTATTGCCGGAATCCTGACGGTTG
CGAGTCACCATGGTGTTTCACAACGGACCCGAACATCCGGGTCGG
GTACTGCTCTCAGATTCCCAACTGTGATATGAGTCATGGCCAGGA
TTGCTATCGGGGCAATGGCAAGAATTACATGGGGAATCTGAGTCA
GACCCGATCCGGACTGACGTGTTCGATGTGGGACAAGAACATGGA
GGACTTGCACCGTCACATATTCTGGGAGCCTGACGCCAGCAAGCT
CAATGAGAATTATTGTCGGAACCCCGACGATGATGCTCACGGGCC
CTGGTGTTACACGGGTAATCCTTTGATACCGTGGGACTACTGTCC
GATCTCGCGCTGCCAGGGGGACACTACTCCCACGATTGTCAATCT
GGATCACGGGGTGATTTCTTGCGCAAAGACCAAGCAACTCCGTGT
GGTAAATGGGATACCGACGAGGACGAACATCGGCTGGATGGTGTC
CTTGCGGTATCGAAACAAGCACATCTGCGGTGGGTCGCTCATCAA
GGAGAGTTGGGTCCTGGACGGCCCGTCAGTGTTTCCCTTCTCGTG
ATCTGAAAGATTACGAGGGCTGGCTCGGGATACACGACGTACACG
GGGGGGGAGACGAGAAGTGCAAGCAAGTACTTAACGTCTCCCAGC
TGGTGTACGGGCCGGAGGGATCCGACTTGGTCTTGATGAAGCTCG
CTCGCCCAGCTGTGCTCGATGATTTTGTGAGCACTATGGATCTCC
CTAATTACGGGTGCACCATCCCGGAGAAGACCTCTTGTAGTGTTC
TACGGATGGGGCTACACTGGCCTCATCAACTACGACGGTCTTCTC
CGAGTGGCGCACCTGTACATTATGGGGAACGAGAAGTGCTCACAA
CATCATCGCGGCAAGGTCACTCTCAACGAGAGTGAGATTTGTGCT
GGAGCCGAGAAAATCGGTTCTGGTCCATGTGAGGGTGATTATGGG
GGTCCACTGGTATGTGAGCAACACAAGATGAGGATGGTCCTGGGG
GTCATTGTTCCCGGCCGAGGGTGTGCCATACCCAACCGGCCGGGG
ATCTTCGTCCGTGTTGCTTACTATGCCAAGTGGATCCACAAGATC
ATTCTCACGTACAAGGTGCCCCAGAGC
SEQ ID NO. 30:
ATGTGGGTGACCAAGCTGTTACCAGCTCTGTTACTGCAGCATGTC
TTGCTTCATCTCTTGCTCTTGGCTATCGCCATCCCTTACGCTGA
GGGTCAGGGTAAGCGTAGGAACACGATCCATGAATTCAAGAAGTC
TGCAAAGACTACTTTAATCAAGATCGATCCTGCGCTCAAGATAAA
GACAAAGAAGGTCAACACGGCCGATCAGTGTGCCAACAGGTGCAC
CGGCAACAAGGGGTTGCCGTTCACCTGTAAGGCCTTTGTCTTCGA
CAAGGCCCGTAAGCAGTGCCTGTGGTTCCCTTTTAACTGCATGTC
CAGTGGAGTTAAGAAGGAATTGGGGCACGAATTTGGATGTTTATG
AAAATAAAGATTACATTCGTAACTGCATTATCGGGAAGGGGCGCT
CGTACAAGGGAACCGTATCGATCACCAAGTCTGGCATCAAATGCC
AGCCTTGGTCATCGATGATTCCTCACGAGCACTCCTTCCTGCCTT
CCTCCTACCGCGGGAAGGATCTCCAGGAGAATTACTGCCGTAATC
CTCGTGGAGAGGAGGGAGGGCCTTGGTGTTTTACTTCGAACCCCG
AGGTAAGATACGAGGTGTGCGATATCCCGCAGTGCTCGGAGGTCG
AATGCATGACATGCAACGGCGAAAGTTACCGTGGCCTGATGGATC
ATACCGAGAGCGGTAAGATCTGTCAGCGGTGGGATCACCAGACCC
CTCACCGTCACAAATTTTTGCGGGAGCGGTACCCCGACAAGGGTT
TCGACGATAACTATTGTCGAAACCCTGACGGGCAGCCGCGTCCGT
GGTGCTACACCCTGGATGCTCACACCCGTTGGGAGTACTGCGCAA
TAAAGACTTGGGCAGATAACACGATGAACGACACCGACGTTCCCC
TGGAGACCACGGACTGCATACAGGGGCAGGGGGAGGGCTACCGCG
GCACGGTGAATACCATTTGGAATGGTATTGCTTGCCAACGGTGGG
ACTCCCAATACCCCCATGAGCACGACATGACTCCAGAGAACTTCA
AGTGTAAGGATCTGGGCGAGAATTATTGCAGGAACCCCGACGGGA
GTGAGAGTCCATGGTGTTTCACTACGGACCCGAACATTCGGGTTG
GCTACTGTAGCCAGATCCCGAATTGCGACATGAGCCATGGGCAAG
ACTGTTATCGGGGTAACGGCAAGAATTACATGGGCAACTTGTCGC
AGACCCGCTCGGGTCTCACATGCAGCATGTGGGACAAGAATATGG
AGGATCTCCATCGTCACATATTCTGGGAGCCGGATGCGTCCAAGT
TGAATGAGAATTATTGCCGTAACCCCGATGACGATGCCCATGGCC
CATGGTGCTATACGGGAAACCCACTCATACCGTGGGATTACTGCC
CGATCTCACGGTGTGAGGGGGACACCACCCCAACCATTGTTAATC
TGGATCACGCTGTCATTAGCTGTGCGAAGACTAAGCAGCTTCGTG
TGGTTAATGGCATCCCGACCCGGACTAACATTGGTTGGATGGTGT
CTCTCAGATATCGCAACAAGCATATCTGTGGGGGATCTCTTATCA
AGGAAAGTTGGGTCCTCACGGCCCGTCAGTGTTTCCCTTCTGGTG
ATCTGAAAGATTACGAGGCCTGGCTCGGGATACACGACGTACACG
GGCGGGGAGACGAGAAGTGCAAGCAAGTACTTAACGTCTCCCAGC
TGGTGTACGGGCCTGAGGGTTCTGACCTGGTACTAATGAAGCTGG
CGCGGCCAGCTGTATTGGACGACTTCGTCAGCACCATCGATTTGC
CAAATTATGGCTGCACTATCCCTGAGAAGACATCCTGCAGTGTCT
ACGGTTGGGGGTATACGGGGCTCATCAACTATGATGGGCTCCTAC
GCGTGGCACACCTATACATTATGGGTAACGAGAAATGTTGTCAGC
ACCACCGCGGGAAGGTCACTCTCAACGAGAGTGAGATGTGTGCTG
GTGCTGAGAAGATTGGCTCGGGTCCCTGCGAGGGTGATTACGGAG
GACCTCTTGTCTGTGAGCAACACAAGATGCGGATGGTCCTCGGGG
TGATCGTCCCTGGCAGGGGCTGTGCCATTCCCAACCGTCCCGGTA
TTTTTGTCCGTGTGGCGTACTACGCCAAATGGATACATAAGATCA
TCCTGACTTATAAAGTACCACAGAGC
SEQ ID NO. 31:
ATGTGGGTGACCAAGTTGCTGCCGGCGCTGTTGTTGCAGCATGTT
CTGCTGCATCTGCTGCTCCTCCCCATCGCGATCCCGTATGCGGA
GGGGCAGCGGAAGCGGCGGAACACGATCCATGAGTTCAAGAAGTC
TGCGAAGACTACGTTGATCAAGATCGATCCGGCGTTGAAGATCAA
GACCAAGAAGGTCAACACGGCGGATCAGTGCGCCAACCGGTGCAC
TCGGAACAAGGGGCTGCCGTTCACGTGTAAGGCCTTTGTTTTCGA
CAAGGCTGGGAAGCAGTGCCTGTGGTTCCCTTTTAACTCCATGAG
CAGTGGGGTTAAGAAGGAATTCGGGCATGAGTTCGACCTGTACGA
GAACAAGGACTATATACGGAACTGCATCATCGGGAAGGGCAGGTC
CTACAAGGGCACGGTGAGCATCACGAAGAGGGGCATCAAGTGCCA
GCCTTGGAGCAGCATGATACCGCACGAGCACAGCTTGCTGCCCTG
CTGCTACCGGGGGAAGGACCTGCAGGAGAACTACTGCAGGAACCC
CCGGGGGGAGGAGGGCGGGCCTTGGTGCTTCACCTCCAACCCGGA
GGTGAGGTACGAGGTGTGGGATATGCGGCAGTGCTCAGAGGTGGA
GTGTATGACTTGCAACGGCGAGTCGTACAGGGGTCTGATGGACCA
CACCGAGAGTGGCAAGATTTGCCAGCGGTGGGACCATCAGACCCC
CCACAGGCACAAGTTCCTGCCGGAGCGCTACCCGGACAAGGGTTT
CGACGATAACTATTGTCGGAACCCGGACGGGCAGCCCCGGCCGTG
GTGCTACACTCTTGATCCGCATACCCGGTGGGAGTACTGCGCGAT
CAAGACGTGGGGGGACAACACCATGAACGATACCGACGTCCCCCT
GGAGACCACTGAGTGCATTCAGGGCCAGGGGGAGGGGTATCGGGG
TACTGTGAACACCATCTGGAACGGGATCCCGTGCCAGAGGTGGGA
CTCGCAGTACCCCCACGAGCACGACATGACCCGGGAGAACTTTAA
GTGTAAGGATCTCCGGGAGAATTACTGTCGTAATCCTGACGGGAG
TGAGAGCCCCTGGTGGTTCACGACGGATCCGAACATCCGCGTGGG
GTATTGCAGTCAGATCCCTAACTGCGACATGAGCCATGGGCAGGA
TTGTTATGGGGGTAACGGCAAGAATTACATGGGCAACTTGTCGCA
GACGCGCTCCGGTCTCACGTGCAGCATGTGGGACAAGAATATGGA
GGATCTGCATCGTCACATATTCTGGGAGCCGGATGCGAGCAAGTT
GAACGAGAATTATTGGCGTAACCCCGATGACGACGCCCATGGCCC
GTGGTGCTACACTGGTAACCCCCTCATACCGTGGGATTACTGCCC
GATCTCGCGGTGTGAGGGGGATACCACGCCCACGATCGTTAATCT
GGACCACCCCGTGATATCTTGTGCGAAGACGAAGCAGCTCCGTGT
GGTCAATGGTATTCCGACGCGGACTAACATTGGCTGGATGGTGTC
GCTTCGTTATCGCAACAAGCATATCTGCGGGGGGTCGCTGATTAA
GGAGTCGTGGGTGCTGACGGGGAGGCAGTGCTTCGCCTCGCGTGA
TCTGAAGGATTACGAGGCCTGGCTGGGCATCCACGATGTCCACGG
GCGTGGGGATGAGAAGTGTAAGCAGGTGCTTAACGTTTGTCAGCT
CGTGTACGGCCCGGAGGGCTCTGACCTGGTGTTGATGAAGTTGGC
CCGCCCTGCCGTTCTCGATGACTTTGTTTCGACTATCGATCTCCC
TAATTACGGGTGCACCATGGGGGAGAAGACCAGCTGTAGCGTCTA
CGGCTGGGGCTATACGGGTTTGATAAACTACGATGGCCTTCTCCG
GGTGGCGCACCTGTACATTATGGGGAACGAGAAGTGTTCTCAGCA
CCACCGCGGGAAGGTCACTCTCAACGAGAGTGAGATCTGCGCTGG
TGCTGAGAAGATGGGGTCGGGGCCCTGCGAGGGCGACTACGGGGG
ACCGCTGGTCTGCGAGCAGCATAAGATGAGGATGGTCTTGGGGGT
GATTGTCCCCGGCCGGGGGTGTGCGATACCCAACCGGCCGGGGAT
ATTCGTCCCCGTGGGGTACTACGCCAAGTGGATCCACAAGATCAT
CCTCACTTATAAGGTGCCGCAGTCG

### EXAMPLE 2 PREPARATION OF mRNA

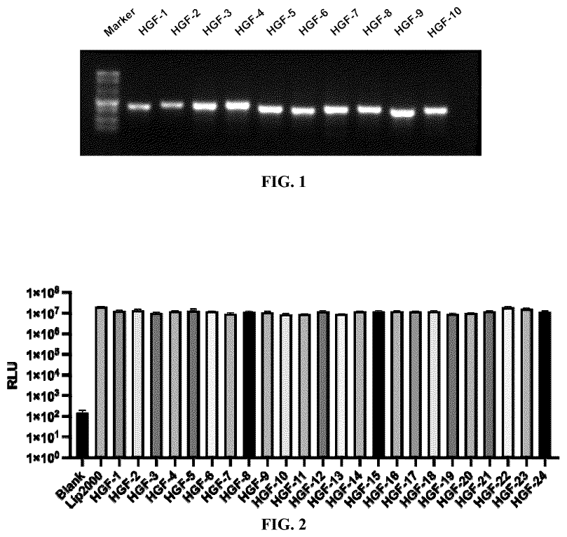

DNA plasmids containing HGF encoding sequences were linearized, and then subjected to in vitro transcription to obtain mRNA. The in vitro transcription product mRNA was detected by agarose gel electrophoresis, and a strip that is bright, single and free from tailing and has good integrity can be seen (FIG. 1). The typical preparation process is as follows.

### 2.1 Digestion of Plasmid DNA via Restriction Endonuclease Reagents for digestion are as shown in Table 4.

TABLE 4
Reagents for digestion

| Name | Volume | |
|---|---|---|
| speI-HF | 1 | μl |
| (NEB) | | |
| Plasmid DNA | 10 | μg |
| 10 × CutSmart buffer (NEB) | 2 | μl |

| | |
|---|---|
| Nuclease-free water | Supplement to a total volume of 20 μl |

The above reagents were evenly mixed by vortex, instantly centrifuged for 10 s in a centrifuge and then reacted for 3 h in a metal bath at 37° C. If the amount of the added plasmid DNA was changed, the amounts of other additives were correspondingly adjusted.

### 2.2 Precipitation of Plasmid DNA

(1) 0.7-fold volume of isopropanol was added into a sample after reaction and then sufficiently and evenly mixed.
(2) DNA was precipitated after being centrifuged for 15 min at 13000 rpm at room temperature. The supernatant was sucked away, then 1 ml of 70% ethanol was added to wash the obtained precipitate, the washed precipitate was centrifuged for 10 min at 13000 rpm at room temperature, and subsequently the supernatant was removed.
(3) After the supernatant was removed, centrifuging was performed for 10 s so that residual ethanol on the wall of a centrifugal tube was collected to the bottom of the tube and then sucked away with a sucker, and then the centrifugal tube was placed for 2 min at room temperature to blow ethanol to be dryness. 50 μL of ultrapure water was added to dissolve the DNA precipitate.
(4) After the precipitate was completely dissolved, the concentration of DNA was detected using NanoDrop (Thermo Fisher), and then the plasmid DNA was stored in a refrigerator at −20° C.

### 2.3 In Vitro Transcription

An in vitro transcription kit of novoprotein is used for in vitro transcription, and its article number is E131-01A.
(1) 10×IVT Buffer was thawed at room temperature, 100 nM NTP and Cleancap were thawed on ice, and T7 Enzyme Mix was thawed in an ice box.
(2) The following components were successively added into a 1.5 ml clean EP tube (20 μl was taken as an example), as shown in Table 5.

TABLE 5
Each component added into the tube

| Name | Volume (μl) |
|---|---|
| IVT Reaction buffer | 2 |
| 100 mM ATP | 1.5 |
| 100 mM CTP | 1.5 |
| 100 mM pUTP | 1.5 |
| 100 mM GTP | 1.5 |
| DNA | 1 μg |
| T7 Enzyme Mix | 1 |
| Nuclease-free water | Supplement to a total volume of 20 μl |

(3) The components were evenly mixed for 5 s by vortex, centrifuged for 10 s in a centrifuge and then incubated for 2-3 h in a constant-temperature mixer at 37° C.
(4) After the reaction was ended, 1 μl of DNaseI was added and the mixture was incubated for 15 min at 37° C., and then a linearized template was removed.

### 2.4 Purification of mRNA

(1) RNA Clean Beads were taken out from 2-8° C. 30 min ahead and then balanced to room temperature, and the beads were evenly mixed by inverted or vortex oscillation.
(2) 36 μl of RNA Clean Beads were added into a mixed solution subjected to IVT.
(3) The mixed solution was blown with a pipettor to be fully mixed.
(4) The above mixture was incubated for 5 min at room temperature so that RNA was bound to the beads.
(5) A sample was placed on a magnetic rack for 5 min, and the supernatant was carefully removed after the solution was clarified.
(6) The sample was kept to be always in the magnetic rack, and the beads were rinsed by adding 200 μl of freshly prepared 80% ethanol and then incubated for 30 s at room temperature.
(7) The supernatant was removed, the former step was repeated, and the beads were rinsed twice in total.
(8) The sample was kept to be always in the magnetic rack, and the cover was opened to dry the beads with air for 5-10 min.
(9) The sample was taken out from the magnetic rack, a proper volume of nuclease-free water was added, the above materials were blown with the pipettor for 10 times to be evenly mixed, and the obtained mixture was subjected to standing for 5 min at room temperature.
(10) The sample was placed on the magnetic rack for 5 min, and the supernatant was carefully transferred into a new centrifugal tube after the solution was clarified.
(11) The RNA concentration and OD260/280 value were detected using NanoDrop, marks were made, and then the samples were stored in a refrigerator at −80° C.

### EXAMPLE 3 In VITRO TRANSFECTION OF mRNA

### 3.1 Cell Seeding

Human skeletal muscle cells (HSMC) in good growth condition were selected and digested with trypsin for 2 min at 37° C., an HSMC complete culture medium (Gibco) was added to stop digestion, the cells were centrifuged for 5 min at 1000 rpm and then re-suspended with the HSMC complete culture medium, the density of the cells were adjusted to 1×10⁵/mL, and subsequently the cells were inoculated into 48-well cell culture plate in an amount of 200 μL/well.

### 3.2 Preparation of Transfection Mix

HGF mRNA and luciferase mRNA (Trilink, article No. L-7202) were respectively diluted to 200 ng/μL with RNase-free water.

(1) Preparation of mRNA Solution

100 ng of HGF mRNA and 100 ng of luciferase mRNA were taken from each well into a centrifugal tube, then 20 μL of Opti MEM™ (Gibco) culture medium was added, and then the above materials were sufficiently and evenly mixed.

### (2) Preparation of Lipofectamine 2000 Solution

0.6 μL of Lipofectamine 2000 (Thermo Fisher) transfection reagent was taken from each well, 20 μL of Opti-MEM culture medium was added, and then the above materials were sufficiently and evenly mixed;

### (3) Preparation of Transfection Compound Mixture

The mRNA diluent was added into the Lipofectamine 2000 diluent to be sufficiently and evenly mixed, and the obtained mixture was subjected to standing for 10 min at room temperature.

### 3.3 mRNA Transfection

160 μL of Opti-MEM culture medium was added into the transfection complex to be sufficiently and evenly mixed, the original culture medium in the cell culture plate was sucked away, and 200 μL of prepared transfection compound mixture was added into each well.

### 3.4 Replace of the Culture Medium

After 4 hours of transfection, the cell was observed, and the culture medium in the cell culture plate was replaced with 200 μL of HSMC complete cell medium.

### 3.5 Collection of ELISA Samples

After 24 hours of transfection, the supernatant in the cell culture plate was collected to detect the expression of HGF;

The cells in the cell culture plate were collected for detection the expression level of luciferase.

### EXAMPLE 4 DETECTION OF EXPRESSION LEVEL OF LUCIFERASE

The expression of luciferase was detected using a luciferase reporter gene detection system (Promega, article number E1501).

### 4.1 Preparation of Reagents

### (1) 1-Fold Cell Lysate

The amounts were calculated in advance, and 5-fold cell lysate was diluted into 1-fold application working solution with deionized water.

### (2) Preparation of Luciferase Detection Solution

The luciferase detection buffer solution was taken from −20° C. and completely thawed, and then the thawed luciferase detection buffer solution was added into a luciferase detection substrate to be completely dissolved so as to obtain the luciferase detection solution.

### 4.2 Test Steps

(1) After transfection, the culture medium was sucked away and the cells were washed with 1×PBS;
(2) 50 μL of 1-fold cell lysate was added into the cell culture plate and then placed for 10 min at room temperature to lyse the cells.
(3) 20 μL of cell lysates were successively taken and transferred into an elisa plates with opaque bottoms to be quickly and evenly mixed, and then chemiluminiscence values were detected by ELISA.

### 4.3 Detection Results

The detection results showed that after the luciferase mRNA was transfected into HSMC cells, the luciferase expression difference of cells was not significant (FIG. 2), and therefore the luciferase can be used as an internal reference for correcting the HGF expression level.

### EXAMPLE 5 IDENTIFICATION OF HGF EXPRESSION LEVEL

### 5.1 Preparation of Reagents

### (1) Warming of Reagent Kit

The HGF detection kit (Solarbio, article number SEKH-0201) was warmed at room temperature.

### (2) Preparation of Washing Solution

The use volume required for a diluted washing solution was calculated, 20×concentrated washing solution was diluted into 1×application solution with deionized water.

### (3) Gradient Dilution of Standards

1 mL of standard diluent was added into lyophilized standards, and then slightly and evenly shaken after being completely dissolved (the concentration was 8000 pg/mL), and the standard diluent was used for gradient dilution according to the following concentrations: 8000, 4000, 2000, 1000, 500, 250, 125 and 0 pg/mL.

### (4) Biotinylated Antibody Working Solution

The amounts required for test were calculated in advance, and a 100×antibody concentrated solution was diluted into 1×application working solution with a detection diluent (SR2) and then added into reaction wells within 30 min.

### (5) Enzyme Conjugate Working Solution

The amounts required for test were calculated in advance, and a 100×enzyme conjugate concentrated solution was diluted into 1×application working solution with an enzyme conjugate diluent (SR3) and then added into reaction wells within 30 min.

### 5.2 Detection Steps

(1) After the kit was warmed to room temperature, the cell culture supernatant sample was diluted with a sample diluent, the plate was taken out, washed with a washing solution for 3 times and then spin-dried;
(2) the standard and the detection sample were added into the reaction wells, and then incubated in an incubator at 37° C. for 90 min after the plate was sealed;
(3) the solution in the elisa plate was swung and then dried with water absorbing paper, and 300 μL of washing solution was added in each well, the interval between the addition time and the plate swing time was 30 s, and the plate was washed 4 times;
(4) 100 μL of biotinylated antibody working solution was added into the reaction well, and then incubated in an incubator at 37° C. for 60 min after the plate was sealed;
(5) the plate washing step in step (3) was repeated;
(6) 100 μL of enzyme conjugate working solution into the reaction well, and then incubated in an incubator at 37° C. for 30 min after the plate was sealed;
(7) the plate washing step in step (3) was repeated for 5 times;
(8) 100 μL of developing substrate added into the reaction well, and developing was performed for 15 min under the condition of dark at 37° C. after the plate was sealed;
(9) 50 μL of developing substrate was added into the reaction well, and dual-wavelength detection was performed by ELIASA within 5 min, and a measurement value was obtained by subtracting the OD value at 630 nm from the OD value at 450 nm;
(10) a standard curve was plotted with Excel based on the concentration as the abscissa and the OD450-0D630 value as the ordinate, and then the content of HGF in the sample was calculated by using the standard curve through the corresponding OD value.

### 5.3 Calculation of Absolute Expression Level

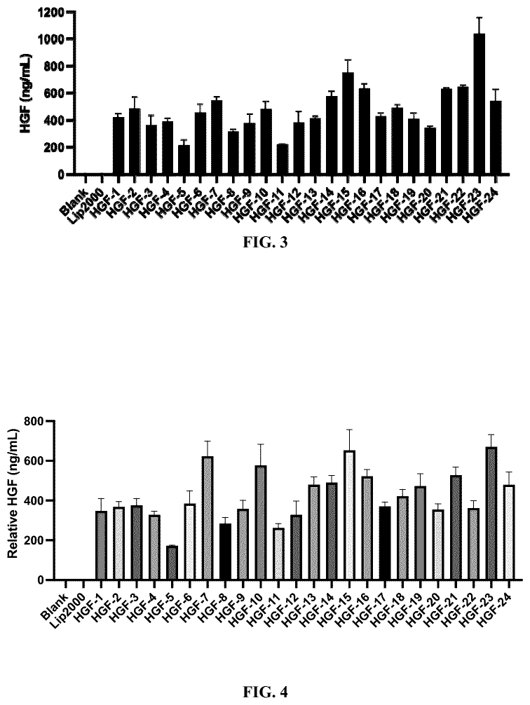

The formula of the standard curve is as follows: y=0.0002x+0.0028 (R²=0.9987). The detection results show that after different HGF mRNA are transfected into HSMC cells, the HGF expression levels in the culture supernatant are increased to a varying degrees (FIG. 3), and the HGF concentration is 200-1040 ng/mL, wherein the content of HGF in the HGF-23 mRNA sample is the highest, and the secretion of HGF in the cell culture supernatant is 1040 ng/mL.

### 5.4 Calculation of Relative Expression Level

Since the addition amount of mRNA, quantity and status of cells and transfection efficiency affect the expression of HGF in the process of experimental reaction, in order to improve the accuracy of experimental data, the expression of luciferase mRNA is selected as internal reference, the RLU value of the HGF-10 sample is used as 1 unit, and the expression level of HGF is corrected to obtain the relative expression level of HGF. The calculation formula of each sample is as follows:

$$Relative expression level of H G F ( ng / mL ) = \frac{HGFn}{RLUn} \times RLU 10 \qquad Formula ( 1 )$$

Wherein, HGFn represents an HGF expression level corresponding to the nth HGF sequence sample (ng/mL); RLUn represents bioluminescence intensity corresponding to the nth HGF sequence sample, representing the expression level of luciferase.

The results show that the relative expression level of HGF in the corrected cell culture supernatant is 173-670 ng/mL (FIG. 4), which can better reflect the expression difference between different HGF mRNA sequences. The artificially designed sequences HGF-7, 10, 13, 14, 15, 16, 18, 19, 21, 23 and 24 have better protein expression levels superior to those of natural sequences (HGF-1 and 2); wherein, the protein expression levels of HGF-7 (the nucleic acid sequence of the obtained construct is SEQ ID No.32), HGF-10 (the nucleic acid sequence of the obtained construct is SEQ ID No.33), HGF-15 (the nucleic acid sequence of the obtained construct is SEQ ID No.34) and HGF-23 (the nucleic acid sequence of the obtained construct is SEQ ID No.35) are more superior to those of other sequences.

The nucleotide sequences of the optimal four nucleic acid constructs are as follows:

SEQ ID NO. 32:
TAATACGACTCACTATAAGGAAATAAGAGAGAAAAGAAGAGTAAG
AAGAAATATAAGAGCCACCATGTGGGTAACAAAATTGCTACCTG
CATTGCTGCTACAGCATGTTCTGCTGCATCTGCTGCTCCTCCCCA
TAGCGATCCCCTATGCGGAGGGGCAGCGGAAGCGGCGGAACACCA
TACATGAGTTTAAGAAGTCTGCGAAGACTACTCTCATCAAAATCG
ATCCCGCCCTGAAGATAAAGACGAAGAAGGTGAATACCGCAGACC
AGTGCGCGAACCGTTGTACGAGGAACAAAGGCCTGCCGTTCACGT
GCAAGGCCTTTGTCTTCGACAAGGCTCGCAAGCAGTGTCTGTGGT
TTCCCTTCAACTCTATGTCTTCAGGGGTGAAGAAGGAATTGGGAC
ACGAATTCGATCTTTACGAAAACAAGGACTATATACGTAATTGTA
TAATTGGGAAGGGGCGCTCGTATAAGGGAACAGTCTCGATCACGA
AGTCTGGCATAAAGTGCCAGCCGTGGTCGAGTATGATCCCTCACG
AGCACTCCTTCCTGGCCTCCTCCTACCGGGGGAAAGACCTGCAGG
AAAATTACTGCAGGAACCCCCGGGGGGAGGAGGGCGGTCCTTGGT
GCTTCACCTCCAACCCTGAGGTGCGTTACGAAGTTTGTGACATAC
CTCAGTGTTCGGAGGTGGAGTGCATGACCTGTAACGGGGAGTCTT
ACCGGGGTCTGATGGACCACACCGAGTCTGGCAAAATTTGCCAGC
GGTGGGACCATCAGACCCCCCACCGGCATAAATTTTTACCTGAGC
GGTACCCCGATAAGGGTTTCGACGATAACTATTGTCGAAACCCTG
ACGGGCAGCCGCGTCCGTGGTGTTACACGTTGGATCCGCACACGC
GATGGGAGTACTGTGCAATCAAGACGTGTGGGGATAATACGATGA
ACGATACGGACGTGCCCCTGGAGACCACGGAGTGTATCCAGGGGC
AGGGTGAAGGGTATCGGGGGACAGTGAACACCATCTGGAATGGGA
TCCCGTGTCAGCGCTGGGACTCTCAGTACCCGCATGAGCACGACA
TGACGCCGGAGAACTTTAAATGTAAAGATCTCCGGGAGAATTACT
GTCGTAATCCTGACGGGTCTGAGAGTCCCTGGTGCTTCACGACGG
ATGCCAACATCCGGGTGGGTTACTGTTCCCAGATACCCAACTGTG
ACATGAGTCACGGTCAGGATTGTTATCGGGGTAACGGCAAGAATT
ACATGGGCAACTTGTCGCAGACGGGCTCCGGTCTCACATGCAGCA
TGTGGGACAAGAATATGGAGGATCTACATCGTCACATATTCTGGG
AGCCGGATGCGAGCAAGTTGAACGAGAATTATTGCCGTAACCCCG
ATGACGATGCTCATGGTCCCTGGTGTTATACGGGAAACCCGTTGA
TACCATGGGACTATTCCCCGATTTCTCGTTGCGAGGGGGATACGA
CGCCCACGATCGTTAATCTGGACCACCCCGTGATATCTTGTGCGA
AGACGAAGCAGCTCCGTGTAGTCAATGGGATTCCGACACGGACTA
ACATTGGCTGGATGGTGTCGCTTCGTTATCGCAACAAGCATATCT
GCGGGGGGTCCTTGATTAAGGAATCGTGGGTGCTGACGGCCGGTC
AGTGTTTCGCCAGCCGGGACCTCAAGGATTATGAGGGCTGGCTGG
GGATACACGACGTACACGGGCGGGGGGACGAGAAGTGCAAACAAG
TACTTAACGTCTCCCAGCTCGTGTACGGGCCGGAGGGATCAGATC
TCGTTCTCATGAAATTAGCGCGACCTGCTGTCCTCGATGATTTTG
TGAGCACTATCGATCTCCCTAATTACGGGTGCACCATCCCGGAGA
AGACCAGTTGTAGTGTATATGGATGGGGCTACACTGGCCTCATCA
ACTACGACGGTCTTCTCCGGGTGGCGCACCTGTATATTATGGGGA
ACGAGAAGTGCTCACAACATCATCGAGGCAAGGTCACGCTGAATG
AGAGCGAGATCTGCGCAGGGGCCGAGAAGATAGGTTCCGGCCCCT
GCGAGGGGGACTACGGTGGTCCCCTTGTATGTGAGCAGCATAAGA
TGAGGATGGTCTTGGGAGTGATTGTCCCCGGCCGGGGGTGTGCAA
TACCCAACCGGCCGGGGATATTCGTCCGCGTGGCGTACTACGCCA
AGTGGATCCACAAGATCATCCTCACTTATAAGGTTCCACAGTCCT
GATAATAGGCTGGAGCCTCGGTGGCCATGCTTCTTGCCCCTTGGG
CCTCCCCCCAGCCCCTCCTCCCCTTCCTGCACCCGTGCTTCTTGC
CCCTTTGAATAAAGTCTGAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAA
SEQ ID NO. 33:
TAATACGACTCACTATAAGGAAATAAGAGAGAAAAGAAGAGTAAG
AAGAAATATAAGAGCCACCATGTGGGTGACAAAGTTGTTACCGG
CCCTGTTACTTCAGCATGTCTTGCTTCACTTGCTTCTGTTGCCGA
TCGCCATTCCTTACGCTGAGGGTCAGCGTAAGCGCCGGAACACGA
TCCATGAATTCAAGAAGTCTGCGAAGACTACCTTGATCAAGATCG
ATCCGGCGCTAAAAATCAAGACTAAGAAGGTGAATACCGCAGACC
AGTGCGCGAACCGTTGTACGAGGAACAAGGGCCTGCCGTTCACGT
GCAAGGCCTTTGTCTTCGACAAGGCTCGCAAGCAGTGTCTGTGGT
TTCCCTTCAATAGTATGAGCTCGGGGGTCAAGAAGGAGTTCGGGC
ATGAGTTCGATCTCTACGAGAACAAGGACTACATCCGGAACTGCA
TCATTGGGAAGGGGCGCTCGTACAAGGGGACCGTGTCGATCACGA
AGTCTGGCATCAAGTGCCAGCCGTGGTCGAGCATGATCCCTCACG
AGCACTGCTTCTTGCCCTCGAGCTACCGCGGGAAGGACCTGCAGG
AGAATTACTGCAGGAACCCCCGCGGTGAGGAGGGCGGTCCATGGT
GCTTCACCAGCAATCCGGAGGTCAGGTACGAGGTATGCGACATAC
CTCAGTGCTCTGAGGTGGAGTGCATGACATGTAACGGCGAGAGTT
ACAGGGGCCTGATGGATCATACCGAATCCGGCAAGATTTGTCAGG
GGTGGGATCACCAGACTGCGCACGGCCACAAGTTCTTGCCGGAGC
GGTATCCGGATAAGGGTTTCGACGATAACTATTGTCGAAACCCTG
ACGGTCAGCCTCGTCCATGGTGCTACACGCTTGATCCGCATACCC
GGTGGGAGTACTGCGCGATCAAGACGTGTGCCGATAACACCATGA
ACGACACTGACGTCCCATTGGAGACAACCGAGTGCATACAGGGAC
AGGGCGAGGGTTATCGGGGTACTGTGAACACCATGTGGAACGGGA
TCCCGTGCCAGAGGTGGGATTCACAGTACCCCCACGAGCATGACA
TGACTCCTGAGAATTTCAAGTGCAAGGATCTGCGAGAGAACTACT
GTCGGAACCGGGACGGTTCGGAGTCGCCGTGGTGCTTCACCACGG
ACCCGAACATCGGGGTGGGGTACTGTTCGCAGATCCCCAACTGCG
ACATGTCTCACGGGCAGGACTGTTACCGGGGGAACGGGAAGAACT
ATATGGGCAACCTGTCCCAGACACGGAGCGGGCTAACCTGCTCCA
TGTGGGATAAGAATATGGAGGACCTCCATAGGCATATCTTCTGGG
AGCCGGATGCCTCTAAGCTCAATGAGAATTATTGCCGGAACCGGG
ATGATGATGCTCATGGGCCGTGGTGTTATACGGGGAACCCGTTGA
TACCGTGGGACTACTGTCCCATCTCGCGATGCGAGGGGGACACTA
CTCCCACTATAGTTAACCTGGACCACCCGGTGATATGCTGCGCGA
AGACAAAGCAGTTGCGGGTGGTGAACGGTATCCCCACCGGGACCA
ACATAGGTTGGATGGTCAGCCTCGGCTACAGAAATAAGCATATCT
GTGGCGGGTCGCTGATCAAGGAGTCATGGGTTCTGACAGCCAGGC
AGTGCTTTCCGTCCCGCGACTTGAAGGACTACGAGGCCTGGTTGG
GCATTCATGACGTACACGGGGGGGGAGACGAGAAGTGCAAGCAAG
TACTTAACGTCTCCCAGCTGGTGTACGGTCCTGAAGGGTCCGACC
TGGTCTTGATGAAGCTCGCCCGCCCTGCTGTATTGGATGACTTCG
TGTCAACCATAGATTTGGGGAACTATGGTTGCACGATCCCTGAGA
AGACTAGTTGCTCGGTGTACGGGTGGGGCTACACAGGGCTCATCA
ACTATGATGGGCTGCTGGGCGTAGCGCACCTGTACATCATGGGCA
ACGAGAAGTGTAGCCAGCATCACCGGGGCAAGGTCACTCTCAACG
AGAGTGAGATTTGCGCCGGTGCCGAGAAAATCGGCAGCGGCCCCT
GCGAGGGCGATTACGGAGGGCCCCTCGTCTGTGAGCAGCACAAGA
TGAGGATGGTCCTCGGGGTGATCGTGCCTGGCAGGGGCTGCGCGA
TACCTAATCGTCCGGGGATCTTCGTCCGCGTGGCGTACTACGCCA
AGTGGATACATAAGATCATCCTGACGTATAAGGTACCGCAGAGCT
GAGCTCGCTTTCTTGCTGTCCAATTTCTATTAAAGGTTGCTTTGT
TCCCTAAGTCCAACTACTAAACTGGGGGATATTATGAAGGGCCTT
GAGCATCTGGATTCTGCCTAATAAAAAACATTTATTTTCATTGCG
CTCGCTTTCTTGCTGTCCAATTTCTATTAAAGGTTCCTTTGTTCC
CTAAGTCCAACTACTAAACTGGGGGATATTATGAAGGGCCTTGAG
CATCTGGATTCTGGCTAATAAAAAACATTTATTTTCATTGCAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAA
SEQ ID NO. 34:
TAATACGACTCACTATAAGGAAATAAGAGAGAAAAGAAGAGTAAG
AAGAAATATAAGAGCCACCATGTGGGTGACCAAGCTCCTGCCGG
CCCTGCTGCTCCAGCATGTCCTTCTGCATCTCCTGCTGCTACCCA
TAGGGATCCCCTATGCAGAAGGACAAAGGAAACGCAGAAATACAA
TTCATGAATTCAAGAAATCAGGGAACACTACCCTAATCAAGATAG
ATCCAGCACTGAAGATAAAAACCAAGAAAGTGAATACCGCAGACC
AGTGTGGTAACCGTTGTACGAGGAACAAAGGACTGGCATTCACTT
GCAAGGCTTTTGTTTTTGATAAGGCCCGTAAACAATGCTTGTGGT
TGCCCTTCAACAGCATGTCAAGTGGCGTGAAGAAAGAGTTCGGAC
ACGAATTTGACCTCTATGAGAACAAAGACTACATTAGGAACTGCA
TCATCGGTAAGGGACGCTCGTATAAGGGCACAGTTTCTATCACTA
AGAGTGGCATTAAATGTCAGCCCTGGTCGTCCATGATACCACACG
AACACAGCTTTCTTCCTTCTTCCTATCGCGGAAAGGACTTGCAGG
AAAACTACTGTCGCAATCCGCGAGGGGAAGAAGGGGCACCCTGGT
GCTTCACAAGCAACCCTGAGGTACGCTACGAAGTCTGTGACATTC
CTCAGTGTTCCGAAGTTGAATGCATGACCTGCAACGGGGAGTGGT
ATCGAGGTCTCATGGATCACACAGAATCAGGCAAGATTTGTCAGC
GGTGGGATCACCAGACCCCCCATCGCCACAAATTCTTGCCTGAAC
GATACGCTGACAAGGGTTTTGATGATAATTACTGCCGTAACCCCG
ACGGCCAGCCGAGGCCCTGGTGTTACACTCTTGACCCTCACACCC
GATGGGAGTACTGTGCTATTAAGACGTGCGCGGACAATACTATGA
ATGACACTGATGTGCCTTTGGAGACAACTGAGTGCATACAAGGTC
AAGGCGAAGGCTACCGGGGTACTGTGAATACAATTTGGAATGGGA
TACCATGTCAGAGATGGGATTCGCAGTACCCTCACGAGCATGACA
TGACTCCTGAAAATTTCAAGTGCAAGGACCTACGAGAGAATTACT
GCCGAAATCCAGATGGGTCTGAGAGCCCCTGGTGCTTTACCACTG
ATCCGAACATCAGAGTTGGTTACTGCTCCCAAATACCAAACTGTG
ATATGTGGCACGGACAAGATTGCTATCGGGGGAATGGCAAAAACT
ACATGGGCAACCTGAGTCAAACAAGATCTGGACTAACATGTTCTA
TGTGGGACAAGAACATGGAAGATCTTCATCGTCATATCTTCTGGG
AACCGGATGCAAGTAAGCTGAACGAGAATTACTGCAGAAATCCAG
ACGATGATGCTCACGGACCCTGGTGCTACACGGGAAATCCACTCA
TTCCTTGGGATTACTGCCCCATTTCTGGTTGTGAAGGTGACACCA
CACCTACCATAGTCAACCTGGACCATCCCGTTATATCATGTGCCA
AAACGAAACAATTGCGAGTTGTCAATGGGATCCCAACTCGAACTA
ACATCGGATGGATGGTTTCCCTCAGATACCGTAACAAACATATCT
GCGGGGGATCATTGATCAAGGAGAGTTGGGTTCTTACGGCAAGGC
AGTGTTTCCCTTCGCGAGACTTGAAGGATTACGAAGCTTGGCTTG
GAATTCACGATGTCCACGGAAGAGGAGATGAGAAATGCAAACAGG
TTCTCAATGTTTCGCAGCTTGTATATGGCCCGGAAGGATCAGATC
TGGTGTTAATGAAGTTAGCCAGGCCGGCCGTCCTGGATGATTTCG
TTAGTACAATCGATCTTCCCAATTATGGTTGCACAATCCCGGAGA
AGACCAGTTGTAGCGTCTATGGCTGGGGCTACACTGGATTGATCA
ACTATGATGGGCTATTACCAGTGGCACATCTCTATATAATGGGAA
ATGAGAAATGCTCGCAGCATCACCGAGGGAAGGTGACTCTGAACG
AGTCGGAAATATGTGCTGGGGCCGAGAAGATTGGTTCTGGCCCAT
GTGAGGGGGATTACGGTGGCCCACTGGTTTGTGAGCAACACAAAA
TGAGGATGGTTCTTGGTGTTATTGTTCCTGGTCGGGGATGTGCCA
TTCCAAACCGTCCTGGTATTTTTGTCCGTGTGGCATATTACGCAA
AATGGATACACAAGATTATTCTCACCTATAAGGTACCCCAGTCAT
GATAATAGGCTGGAGCCTCGGTGGCCATGCTTCTTGCCCCTTGGG
CCTCCCCCCAGCCTCCCCTTCCTGCACCCGTACCCCCGTGGTCTT
TGAATAAAGTCTGAAAAAAAAAAAAAAAAAAAAAAAAAAAAAAAG
CATATGACTAAAAAAAAAAAAAAAAAAAAAAAAAAAAAAAAAAAA
AAAAAAAAAAAAAAAAAAAAAAAAAAAAAAAAAAA
SEQ ID NO. 35:
TAATACGACTCACTATAAGGAAATAAGAGAGAAAAGAAGAGTAAG
AAGAAATATAAGAGCCACCATGTGGGTGACCAAGCTGTTACCAG
CTCTGTTACTGCAGCATGTCTTGCTTCATCTCTTGCTCTTGCCTA
TCGCCATCCCTTACGCTGAGGGTCAGCGTAAGCGTAGGAACACGA
TCCATGAATTCAAGAAGTCTGCAAAGACTACTTTAATCAAGATCG
ATCCTGCGCTCAAGATAAAGACAAAGAAGGTCAACACGGCCGATC
AGTGTGCCAACAGGTGCACCGGCAACAAGGGGTTGCCGTTCACCT
GTAAGGCCTTTGTCTTCGACAAGGCCGGTAAGCAGTGCCTGTGGT
TCCCTTTTAACTCCATGTCCAGTGGAGTTAAGAAGGAATTCGGGC
ACGAATTTGATCTTTATGAAAATAAAGATTACATTGGTAACTGCA
TTATCGGGAAGGGGCGCTCGTACAAGGGAACCGTATCGATCACCA
AGTCTGGCATCAAATGCCAGGCTTGGTCATCGATGATTCCTCACG
AGCACTCCTTCCTGCCTTCCTGCTACCGGGGGAAGGATCTCCAGG
AGAATTACTGCCGTAATCCTCGTGGAGAGGAGGGAGGGCCTTGGT
GTTTTACTTCGAACCCCGAGGTAAGATACGAGGTGTGCGATATCC
CGCAGTGCTCGGAGGTCGAATGCATGACATGCAACGGCGAAAGTT
ACGGTGGCCTGATGGATCATACCGAGAGCGGTAAGATCTGTCAGG
GGTGGGATCACCAGACCCCTCACCGTCACAAATTTTTGCCGGAGC
GGTACCCCGACAAGGGTTTCGACGATAACTATTGTCGAAACCCTG
ACGGGCAGCGGCGTCCGTGGTGCTACACCCTGGATCCTCACACCC
GTTGGGAGTACTGCGCAATAAAGACTTGGGCAGATAACACGATGA
ACGACACCGACGTTCCCCTGGAGACCACGGAGTGCATACAGGGGC
AGGGGGAGGGCTACCGCGGCACGGTGAATACCATTTGGAATGGTA
TTCCTTGCCAACGGTGGGACTCCCAATACCCCCATGAGCACGACA
TGACTCCAGAGAACTTCAAGTGTAAGGATCTGCGCGAGAATTATT
GCAGGAACCCCGACGGGAGTGAGAGTCCATGGTGTTTCACTACGG
ACCCGAACATTCGGGTTGGCTACTGTAGCCAGATCCCGAATTGCG
ACATGAGCCATGGGCAAGACTGTTATGGGGGTAACGGCAAGAATT
ACATGGGCAACTTGTCGCAGACCCGCTCCGGTCTCACATGCAGCA
TGTGGGACAAGAATATGGAGGATCTCCATCGTCACATATTCTGGG
ACCCGGATGCGTCCAAGTTGAATGAGAATTATTGCGGTAACCCCG
ATGACGATGCCCATGGCCCATGGTGCTATACGGGAAACCCACTCA
TACGGTGGGATTACTGCCGGATCTCACGGTGTGAGGGGGACACCA
CCCCAACCATTGTTAATCTGGATCACCCTGTCATTAGCTGTGCGA
AGACTAAGCAGCTTCGTGTGGTTAATGGCATCCCGACCCGGACTA
ACATTGGTTGGATGGTGTCTCTCAGATATCGCAACAAGCATATCT
GTGGGGGATCTCTTATCAAGGAAAGTTGGGTCCTCACGGCGCGTC
AGTGTTTCCCTTCTCGTGATCTGAAAGATTACGAGGCCTGGCTCG
GGATACACGACGTACACGGGCGGGGAGACGAGAAGTGCAAGCAAG
TACTTAACGTCTCCCAGCTCGTGTACGGGCCTGAGGGTTCTGACC
TGGTACTAATGAAGCTGGGGGGGCCAGCTGTATTGGACGACTTCG
TCAGCACCATCGATTTGCCAAATTATGGCTGCACTATCCCTGAGA
AGACATCCTGCAGTGTCTACGGTTGGGGGTATACGGGGCTCATCA
ACTATGATGGGCTCCTACGCGTGGCACACCTATACATTATGGGTA
ACGAGAAATGTTCTCAGCACCACCGCGGGAAGGTCACTCTCAACG
AGAGTGAGATCTGTGCTGGTGCTGAGAAGATTGGCTCGGGTCCCT
GCGAGGGTGATTACGGAGGACCTCTTGTCTGTGAGCAACACAAGA
TGCGGATGGTGCTCGGGGTGATCGTCCCTGGCAGGGGCTGTGCCA
TTCCCAACCGTCCCGGTATTTTTGTCCGTGTGGCGTACTACGCCA
AATGGATACATAAGATCATCCTGACTTATAAAGTACCACAGAGCT
GATAATAGGCTGGAGCCTCGGTGGCCATGCTTCTTGCCGCTTGGG
CCTCCCCCCAGCCCCTGCTCCCCTTCCTGCACCGGTACCCCCGTG
GTCTTTGAATAAAGTCTGAAAAAAAAAAAAAAAAAAAAAAAAAAA
AAAAGCATATGACTAAAAAAAAAAAAAAAAAAAAAAAAAAAAAAA
AAAAAAAAAAAAAAAAAAAAAAAAAAAAAAAAAAAAAAAA

The above descriptions are only specific embodiments of the present disclosure, but the protective scope of the present disclosure is not limited thereto. Those skilled in the art can easily think of changes or replacements within the technical scope disclosed by the present disclosure, which shall be contained within the protective scope of the present disclosure. Hence, the protective scope of the present disclosure shall be based on the protective scope of the appended claims.

What is claimed is:

1. A nucleic acid encoding human HGF, the nucleic acid comprising one or more open reading frames (ORFs), wherein the ORF nucleic acid sequence is at least 80%, at least 85%, at least 90%, at least 95%, at least 96%, at least 97%, at least 97%, at least 98%, at least 99% or 100% identical to the nucleic acid sequence of SEQ ID NOs: 21-31.

2. The nucleic acid of claim 1, wherein the ORF nucleic acid sequence is selected from SEQ ID NOs: 21-31, or a transcribed RNA sequence thereof.

3. The nucleic acid of claim 2, wherein the nucleic acid further comprises a 5′cap; preferably, the 5′cap is selected from m7G5′ppp5′Np, m7G5′ppp5′NmpNp and m7G5′ppp5′NmpNmpNp.

4. The nucleic acid of any one of claims 1-3, wherein the nucleic acid further comprises 5′UTR; preferably, the 5′UTR comprises one sequence selected from SEQ ID NOs: 1-8, or a combination thereof.

5. The nucleic acid of any one of claims 1-4, wherein the nucleic acid further comprises 3′UTR; preferably, the 3′UTR comprises one sequence selected from SEQ ID NOs: 9-17, or a combination thereof.

6. The nucleic acid of any one of claims 1, wherein the nucleic acid further comprises a poly-A region comprising 70-150 nucleotides in length; preferably, the poly-A region comprises the sequence set forth in SEQ ID NO: 18 or 19.

7. A construct, the construct comprising the nucleic acid of any one of claims 1-6, wherein the sequence of the construct is selected from SEQ ID NOs: 32, 33, 34, 35, 56, 57, 58, 60, 61, 63 and 65, or a transcribed RNA sequence thereof; preferably, the construct further comprises one or more modified nucleosides selected from pseudouridine, N1-methyl-pseudouridine and 5 -methylcytidine; preferably, the construct is DNA or mRNA.

8. A vector comprising the nucleic acid of any one of claims 1-6.

9. A cell comprising the nucleic acid of any one of claims 1-6.

10. A pharmaceutical composition comprising the nucleic acid of any one of claims 1-6.


SEQUENCE LISTING (35 sequences)

SEQ ID NO: 1 - RNA, 47 nt, synthetic construct
  source 1..47: mol_type = other RNA
aggaaataagagagaaaagaagagtaagaagaaatataagagccacc

SEQ ID NO: 2 - RNA, 41 nt, synthetic construct
  source 1..41: mol_type = other RNA
gggcgaactagtactcttctggtccccacagactcgccacc

SEQ ID NO: 3 - RNA, 132 nt, synthetic construct
  source 1..132: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgatagc

SEQ ID NO: 4 - RNA, 49 nt, synthetic construct
  source 1..49: mol_type = other RNA
aagttgaaagtcgccgctgacagttgtgaccaggatcggacaggtgaac

SEQ ID NO: 5 - RNA, 50 nt, synthetic construct
  source 1..50: mol_type = other RNA
acatttgcttctgacacaactgtgttcactagcaacctcaaacagacacc

SEQ ID NO: 6 - RNA, 37 nt, synthetic construct
  source 1..37: mol_type = other RNA
actcttctggtccccacagactcagagagaacccacc

SEQ ID NO: 7 - RNA, 46 nt, synthetic construct
  source 1..46: mol_type = other RNA
actccccgaaccactcagggtcctgtggacagctcacctagctgca

SEQ ID NO: 8 - RNA, 20 nt, synthetic construct
  source 1..20: mol_type = other RNA
ataaacgctcaactttggcc

SEQ ID NO: 9 - RNA, 101 nt, synthetic construct
  source 1..101: mol_type = other RNA
gctggagcctcggtggccatgcttcttgccccttgggcctccccccagcccctcctccccttcctgcacccgtacccccgtggtctttgaataaagtctga

SEQ ID NO: 10 - RNA, 264 nt, synthetic construct
  source 1..264: mol_type = other RNA
gctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgc

SEQ ID NO: 11 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 12 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 13 - RNA, 507 nt, synthetic construct
  source 1..507: mol_type = other RNA
gcagctcgacgcccgttcgcttggttctgcctgattaccatccagtcgggtgtgggccgttaccacaccggtgaatagttacctgaagcttggtcaaacctggaacatgttggttccacaccttcatatctcaggcagcagaaaaacatgaaggataagtgaaacgcctgcactgataaatcaaagaagagggtaaaatgaaggtcatattttttctgaaaatgcataaataatcttttaaaaatatatatacatactgtatagagagagagagcggtccatggcattattgctgctgagtgacagcttaagttcaacccaggacaggactgctgatccagctgtgctgaatccatttttattgtattaccagaaatacacgttacagtaatgtttttacaatataaacatgagtagttgtgtattttctagaagtttaccgcctcttgttatttgacattagctttctttctcatttattttcttgtaaataaatctcttgtgctc

SEQ ID NO: 14 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 15 - RNA, 111 nt, synthetic construct
  source 1..111: mol_type = other RNA
gctggagcctcggtggccatgcttcttgccccttgggcctccccccagcccctcctccccttcctgcacccgtacccccgtggtctttgaataaagtctgagtgggcggca

SEQ ID NO: 16 - RNA, 107 nt, synthetic construct
  source 1..107: mol_type = other RNA
ctgcccgggtggcatccctgtgacccctccccagtgcctctcctggccctggaagttgccactccagtgcccaccagccttgtcctaataaaattaagttgcatcat

SEQ ID NO: 17 - RNA, 126 nt, synthetic construct
  source 1..126: mol_type = other RNA
accagcctcaagaacacccgaatggagtctctaagctacataataccaacttacactttacaaaatgttgtcccccaaaatgtagccattcgtatctgctcctaataaaaagaaagtttcttcaca

SEQ ID NO: 18 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 19 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 2184 nt, synthetic construct
  source 1..2184: mol_type = other RNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtca

SEQ ID NO: 21 - RNA, 2184 nt, synthetic construct
  source 1..2184: mol_type = other RNA
atgtgggtaacaaaattgctacctgcattgctgctacagcatgttctgctgcatctgctgctcctccccatagcgatcccctatgcggaggggcagcggaagcggcggaacaccatacatgagtttaagaagtctgcgaagactactctcatcaaaatcgatcccgccctgaagataaagacgaagaaggtgaataccgcagaccagtgcgcgaaccgttgtacgaggaacaaaggcctgccgttcacgtgcaaggcctttgtcttcgacaaggctcgcaagcagtgtctgtggtttcccttcaactctatgtcttcaggggtgaagaaggaattcggacacgaattcgatctttacgaaaacaaggactatatacgtaattgtataattgggaaggggcgctcgtataagggaacagtctcgatcacgaagtctggcataaagtgccagccgtggtcgagtatgatccctcacgagcactccttcctgccctcctcctaccgggggaaagacctgcaggaaaattactgcaggaacccccggggggaggagggcggtccttggtgcttcacctccaaccctgaggtgcgttacgaagtttgtgacatacctcagtgttcggaggtggagtgcatgacctgtaacggggagtcttaccggggtctgatggaccacaccgagtctggcaaaatttgccagcggtgggaccatcagaccccgcaccggcataaatttttacctgagcggtaccccgataagggtttcgacgataactattgtcgaaaccctgacgggcagccgcgtccgtggtgttacacgttggatccgcacacgcgatgggagtactgtgcaatcaagacgtgtgcggataatacgatgaacgatacggacgtgcccctggagaccacggagtgtatccaggggcagggtgaagggtatcgggggacagtgaacaccatctggaatgggatcccgtgtcagcgctgggactctcagtacccgcatgagcacgacatgaccccggagaactttaaatgtaaagatctccgggagaattactgtcgtaatcctgacgggtctgagagtccctggtgcttcacgacggatcccaacatccgggtgggttactgttcccagatacccaactgtgacatgagtcacggtcaggattgttatcggggtaacggcaagaattacatgggcaacttgtcgcagacgcgctccggtctcacatgcagcatgtgggacaagaatatggaggatctacatcgtcacatattctgggagccggatgcgagcaagttgaacgagaattattgccgtaaccccgatgacgatgctcatggtccctggtgttatacgggaaacccgttgataccatgggactattgcccgatttctcgttgcgagggggatacgacgcccacgatcgttaatctggaccaccccgtgatatcttgtgcgaagacgaagcagctccgtgtagtcaatgggattccgacacggactaacattggctggatggtgtcgcttcgttatcgcaacaagcatatctgcggggggtccttgattaaggaatcgtgggtgctgacggcccgtcagtgtttccccagccgggacctcaaggattatgaggcctggctggggatacacgacgtacacgggcggggggacgagaagtgcaaacaagtacttaacgtctcccagctcgtgtacgggccggagggatcagatctcgttctcatgaaattagcgcgacctgctgtcctcgatgattttgtgagcactatcgatctccctaattacgggtgcaccatcccggagaagaccagttgtagtgtatatggatggggctacactggcctcatcaactacgacggtcttctccgggtggcgcacctgtatattatggggaacgagaagtgctcacaacatcatcgaggcaaggtcacgctgaatgagagcgagatctgcgcaggggccgagaagataggttccggcccctgcgagggggactacggtggtccccttgtatgtgagcagcataagatgaggatggtcttgggagtgattgtccccggccgggggtgtgcaatacccaaccggccggggatattcgtccgcgtggcgtactacgccaagtggatccacaagatcatcctcacttataaggttccacagtcc

SEQ ID NO: 22 - RNA, 2184 nt, synthetic construct
  source 1..2184: mol_type = other RNA
atgtgggtgacaaagttgttaccggccctgttacttcagcatgtcttgcttcacttgcttctgttgccgatcgccattccttacgctgagggtcagcgtaagcgccggaacacgatccatgaattcaagaagtctgcgaagactaccttgatcaagatcgatccggcgctaaaaatcaagactaagaaggtgaataccgcagaccagtgcgcgaaccgttgtacgaggaacaagggcctgccgttcacgtgcaaggcctttgtcttcgacaaggctcgcaagcagtgtctgtggtttcccttcaatagtatgagctcgggggtcaagaaggagttcgggcatgagttcgatctctacgagaacaaggactacatccggaactgcatcattgggaaggggcgctcgtacaaggggaccgtgtcgatcacgaagtctggcatcaagtgccagccgtggtcgagcatgatccctcacgagcactccttcttgccctcgagctaccgcgggaaggacctgcaggagaattactgcaggaacccccgcggtgaggagggcggtccatggtgcttcaccagcaatccggaggtcaggtacgaggtatgcgacatacctcagtgctctgaggtggagtgcatgacatgtaacggcgagagttacaggggcctgatggatcataccgaatccggcaagatttgtcagcggtgggatcaccagactccgcaccgccacaagttcttgccggagcggtatccggataagggtttcgacgataactattgtcgaaaccctgacggtcagcctcgtccatggtgctacacgcttgatccgcatacccggtgggagtactgcgcgatcaagacgtgtgccgataacaccatgaacgacactgacgtcccattggagacaaccgagtgcatacagggacagggcgagggttatcggggtactgtgaacaccatctggaacgggatcccgtgccagaggtgggattcacagtacccccacgagcatgacatgactcctgagaatttcaagtgcaaggatctccgagagaactactgtcggaacccggacggttcggagtcgccgtggtgcttcaccacggacccgaacatccgggtggggtactgttcgcagatccccaactgcgacatgtctcacgggcaggactgttaccgggggaacgggaagaactatatgggcaacctgtcccagacacggagcgggctaacctgctccatgtgggataagaatatggaggacctccataggcatatcttctgggagccggatgcctctaagctcaatgagaattattgccggaacccggatgatgatgctcatgggccgtggtgttatacggggaacccgttgataccgtgggactactgtcccatctcgcgatgcgagggggacactactcccactatagttaacctggaccacccggtgatatcctgcgcgaagacaaagcagttgcgggtcgtgaacggtatccccacccggaccaacataggttggatggtcagcctccgctacagaaataagcatatctgtggcgggtcgctgatcaaggagtcatgggttctgacagccaggcagtgctttccgtcccgcgacttgaaggactacgaggcctggttgggcattcatgacgtacacgggcggggagacgagaagtgcaagcaagtacttaacgtctcccagctcgtgtacggtcctgaagggtccgacctggtcttgatgaagctcgcccgccctgctgtattggatgacttcgtgtcaaccatagatttgccgaactatggttgcacgatccctgagaagactagttgctcggtgtacgggtggggctacacagggctcatcaactatgatgggctgctgcgcgtagcccacctgtacatcatgggcaacgagaagtgtagccagcatcaccggggcaaggtcactctcaacgagagtgagatttgcgccggtgccgagaaaatcggcagcggcccctgcgagggcgattacggagggccgctcgtctgtgagcagcacaagatgaggatggtcctcggggtgatcgtgcctggcaggggctgcgcgatacctaatcgtccggggatcttcgtccgcgtggcgtactacgccaagtggatacataagatcatcctgacgtataaggtaccgcagagc

SEQ ID NO: 23 - RNA, 2184 nt, synthetic construct
  source 1..2184: mol_type = other RNA
atgtgggtaaccaagttgcttcccgctcttcttctgcagcatgtactgttgcatctgcttctgctgcccatcgctatcccgtacgcggaggggcagcggaagcggcgcaacaccatacatgagttcaagaagagtgcgaagacaactcttatcaagatcgaccccgccctgaagataaagacgaagaaggtgaacactgcagaccagtgtgcgaaccgctgcacgaggaacaaaggtcttccattcacatgcaaggcctttgttttcgacaaggctcgcaagcagtgtctgtggtttcccttcaactctatgtcttcaggtgtgaagaaggaattcggccacgaattcgatctttacgagaacaaggactacatccgtaattgtattatcgggaagggcaggtcttacaagggcacagtctccataacgaagtcggggatcaagtgtcagccatggtcgagtatgattcctcatgagcacagtttccttccttcttcctaccgggggaaggacctgcaggaaaactactgtcgcaacccccgaggggaggagggaggaccttggtgcttcacttcgaaccccgaggtgaggtacgaggtatgcgacatacctcagtgctctgaggtagagtgcatgacgtgcaatggggaatcatacaggggtctgatggatcacaccgagtctggcaagatttgtcaacggtgggatcatcagactcctcatcggcacaagttccttcccgaacggtaccccgacaagggtttcgacgataactattgtcgaaaccctgacgggcagcctcgaccatggtgctacacacttgatccccacactcgttgggagtactgtgccattaagacctgcgccgataatacaatgaacgacacggatgtgccccttgagacgactgagtgcatccagggtcagggtgaagggtatcggggcaccgtgaataccatctggaatgggatcccttgtcagcgctgggactctcagtaccctcacgagcacgacatgactccagagaactttaagtgtaaggatctccgggagaactactgtcgtaatcctgatggttctgagagtccctggtgcttcacaacggatcccaatatcagggttgggtactgcagccagatacctaactgtgatatgagccatgggcaggactgttatcggggtaacggcaaaaattacatgggcaacttgtcgcagacgcgctccgggctgacgtgctcgatgtgggacaaaaacatggaggatctacatcgccacatcttctgggagcccgatgcgtccaagttgaatgagaattattgccgtaaccctgatgacgacgctcatggtccctggtgttacacaggtaaccccctgataccctgggactactgtcctatctcgcgttgtgagggtgacactacccctacgattgtgaatcttgatcaccctgtgatatcgtgtgccaaaacgaagcaactacgcgtagttaatgggatccctacccgcaccaatataggctggatggtgtctctcaggtatcggaacaagcatatttgcggtgggtcgctgataaaggagtcgtgggtcctcacggccaggcagtgttttccttcgagggacttgaaggactacgaggcctggctcgggatccacgacgtccacggacggggggacgagaagtgcaagcaggtcctgaatgtgagtcagctggtctacggaccagagggttcggatttagtcctcatgaagctcgctcgtcctgccgttctggatgacttcgtgtccacgatcgacctaccaaactatggttgtacgatacctgagaagacatcatgcagcgtgtatgggtgggggtacacgggtctcattaactacgacgggttgcttcgtgtggcacatctctatatcatgggcaatgaaaagtgttcacagcaccataggggtaaggtcactctcaacgagagcgagatatgtgctggcgcggagaagattggctccggtccctgcgagggagactacggtggccccctggtctgtgagcagcacaagatgcgcatggtcctgggggtcatagtccctggcaggggctgtgccattcccaaccgtcccggcatatttgttcgcgtggcgtactacgccaagtggatacacaagatcattctgacctacaaggtgcctcagtcg

SEQ ID NO: 24 - RNA, 2184 nt, synthetic construct
  source 1..2184: mol_type = other RNA
atgtgggtcacgaagttgcttcccgctcttcttctgcagcatgtgctgttgcacctgcttctgctgcccatcgctatcccgtacgcggaggggcagcggaagcggcgcaacacgatacatgagttcaagaagagcgcgaagacgactctgatcaagatcgatccggccctgaagataaagacgaagaaggtgaacaccgcggaccagtgtgcgaaccgttgtacgaggaacaagggccttccattcacgtgcaaggcctttgtcttcgacaaggctcgcaagcagtgtctgtggtttcccttcaactctatgtcttcgggcgtgaagaaggagttcgggcacgagttcgatctttacgagaacaaggactacatccgtaattgtattatcgggaagggcaggtcgtataagggcacggtgtctatcaccaagtcggggatcaagtgtcagccctggtcgagtatgattccccatgagcacagtttcctgccctcctcctaccgggggaaggacctgcaggagaactactgcaggaacccccggggggaggagggcgggccttggtgcttcacctcgaaccccgaggtgaggtacgaggtgtgcgacatccctcagtgctctgaggtggagtgcatgacgtgcaatggggagtcgtaccggggtctgatggaccacaccgagagtggcaagatttgccagcggtgggaccatcagacccctcataggcacaagtttcttcctgagaggtacccggacaagggtttcgacgataactattgtcggaacccggacgggcagcctcggccgtggtgctacacgcttgatccccacactcggtgggagtactgtgccattaagacctgcgccgataatacgatgaacgacacggatgtgccgctggagacgactgagtgcatccagggtcagggtgaggggtatcgggggaccgtgaacaccatctggaatgggatcccgtgccagcgctgggactctcagtacccgcacgagcacgacatgactcccgagaactttaagtgtaaggatctgcgggagaattactgtcgtaatcctgacgggtctgagagtccctggtgcttcacgacggatcccaatatcagggttgggtactgcagccagatacccaactgtgatatgagccatgggcaggattgttatcggggtaacggcaagaattacatgggcaacttgtcgcagacgcgctccgggctgacgtgctcgatgtgggataagaacatggaggacttgcatcggcacatcttctgggagcccgacgcgagcaagttgaacgagaattattgccgtaaccccgatgacgacgcccatggcccgtggtgttacacggggaacccgctgataccctgggactactgcccgatctcgcgctgtgagggtgacactacccctacgatcgtgaaccttgatcaccccgtgatatcgtgcgccaagacgaagcagctgcgcgtggttaatgggatccctacccgcaccaatataggctggatggtgtctctcaggtatcgtaacaagcatatttgcggcgggtccctgataaaggagtcgtgggtcctgacggccaggcagtgttttccttcgagggacttgaaggactacgaggcctggctggggatccacgacgtccacgggcggggggacgagaagtgcaagcaggtgctgaatgtgagccagctggtctacgggccggagggctcggatttggtcctcatgaagcttgctcgtcccgccgtcctggacgactttgtcagcacgatcgacctgccgaactatggttgtacgatacctgagaagacgtcgtgcagcgtgtatgggtgggggtacacgggtctcattaactacgacgggctgcttcgcgtggcgcacctgtatatcatggggaatgagaagtgttcgcagcatcatagggggaaggtcaccctcaacgagagcgagatctgtgccggggcggagaagattggcagcggcccctgcgagggggactacggtggcccgctggtatgcgagcagcataagatgcgcatggtgctgggggtcatcgtgcccggcaggggctgcgccattccgaaccgccccggcatctttgtccgcgtggcgtactacgccaagtggatacacaagatcatcctgacctacaaggtgcctcagtcg

SEQ ID NO: 25 - RNA, 2184 nt, synthetic construct
  source 1..2184: mol_type = other RNA
atgtgggtgaccaagctcctgccggccctgctgctccagcatgtccttctgcatctcctcctgctacccatagcgatcccctatgcagaaggacaaaggaaacgcagaaatacaattcatgaattcaagaaatcagcgaagactaccctaatcaagatagatccagcactgaagataaaaaccaagaaagtgaataccgcagaccagtgtgctaaccgttgtacgaggaacaaaggactgccattcacttgcaaggcttttgtttttgataaggcccgtaaacaatgcttgtggttccccttcaacagcatgtcaagtggcgtgaagaaagagttcggacacgaatttgacctctatgagaacaaagactacattaggaactgcatcatcggtaagggacgctcgtataagggcacagtttctatcactaagagtggcattaaatgtcagccctggtcgtccatgataccacacgaacacagctttcttccttcttcctatcgcggaaaggacttgcaggaaaactactgtcgcaatccgcgaggggaagaagggggaccctggtgcttcacaagcaaccctgaggtacgctacgaagtctgtgacattcctcagtgttccgaagttgaatgcatgacctgcaacggggagtcgtatcgaggtctcatggatcacacagaatcaggcaagatttgtcagcggtgggatcaccagaccccccatcgccacaaattcttgcctgaacgataccctgacaagggttttgatgataattactgccgtaaccccgacggccagccgaggccctggtgttacactcttgaccctcacacccgatgggagtactgtgctattaagacgtgcgcggacaatactatgaatgacactgatgtgcctttggagacaactgagtgcatacaaggtcaaggcgaaggctaccggggtactgtgaatacaatttggaatgggataccatgtcagagatgggattcgcagtaccctcacgagcatgacatgactcctgaaaatttcaagtgcaaggacctacgagagaattactgccgaaatccagatgggtctgagagcccctggtgctttaccactgatccgaacatcagagttggttactgctcccaaataccaaactgtgatatgtcgcacggacaagattgctatcgggggaatggcaaaaactacatgggcaacctgagtcaaacaagatctggactaacatgttctatgtgggacaagaacatggaagatcttcatcgtcatatcttctgggaaccggatgcaagtaagctgaacgagaattactgcagaaatccagacgatgatgctcacggaccctggtgctacacgggaaatccactcattccttgggattactgccccatttctcgttgtgaaggtgacaccacacctaccatagtcaacctggaccatcccgttatatcatgtgccaaaacgaaacaattgcgagttgtcaatgggatcccaactcgaactaacatcggatggatggtttccctcagataccgtaacaaacatatctgcgggggatcattgatcaaggagagttgggttcttacggcaaggcagtgtttcccttcgcgagacttgaaggattacgaagcttggcttggaattcacgatgtccacggaagaggagatgagaaatgcaaacaggttctcaatgtttcgcagcttgtatatggcccggaaggatcagatctggtgttaatgaagttagccaggccggccgtcctggatgatttcgttagtacaatcgatcttcccaattatggttgcacaatcccggagaagaccagttgtagcgtctatggctggggctacactggattgatcaactatgatgggctattacgagtggcacatctctatataatgggaaatgagaaatgctcgcagcatcaccgagggaaggtgactctgaacgagtcggaaatatgtgctggggccgagaagattggttctggcccatgtgagggggattacggtggcccactggtttgtgagcaacacaaaatgaggatggttcttggtgttattgttcctggtcggggatgtgccattccaaaccgtcctggtatttttgtccgtgtggcatattacgcaaaatggatacacaagattattctcacctataaggtaccccagtca

SEQ ID NO: 26 - RNA, 2184 nt, synthetic construct
  source 1..2184: mol_type = other RNA
atgtgggtgaccaaactgctgccggccctgctgctgcagcatgtcctcctgcatctcctcctgctgcccatcgccataccctatgccgaggggcagaggaagagacgtaacacgattcatgaattcaaaaaatcagcaaagactactcttatcaagattgatccagcactgaagataaagaccaagaaggtgaacacggcggaccagtgtgccaataggtgtactaggaataagggtctgccgttcacttgcaaggcgtttgtttttgacaaagcgagaaaacaatgcctctggttccctttcaatagcatgtcaagcggggttaagaaggaatttggccacgaatttgacctctatgaaaacaaggactacattcgtaactgtatcattggtaaaggacgaagctacaagggaacagtctctatcactaagagtggcatcaagtgtcagccctggagttcgatgatacctcacgagcacagctttctgccttcgagctatcggggtaaggatcttcaggagaactactgtcgaaatcctcggggggaagaggggggcccctggtgtttcacaagcaatccagaggtgcgctacgaagtctgtgacattccgcagtgttcagaggttgagtgcatgacatgcaatggggagagttatcggggtctcatggatcataccgagtcaggcaagatttgtcagcggtgggatcatcagacgccccaccgccacaaattcttgcctgagcggtatcccgacaagggctttgatgataattattgccggaatccggatggccagccgaggccgtggtgctatactttagaccctcacacccgctgggagtactgtgcaattaagacatgcgctgacaatacgatgaatgacactgatgttcctttggagacaacggaatgtatccaaggtcagggagaaggctacaggggcactgtcaataccatttggaatggaattccatgtcagcggtgggattcgcagtatcctcacgagcatgacatgacgcctgaaaatttcaagtgcaaggacttgcgagagaactactgccgaaatccagacgggtcggaatcaccgtggtgttttaccactgatccgaacatccgagttgggtactgctcccaaattccaaactgcgatatgtcccatgggcaggattgttatcgtgggaatggcaagaattatatgggcaatctgtcccagacaagaagcggattgacgtgtagtatgtgggacaagaacatggaggacctccatcgtcatatcttctgggagccagatgccagtaagctcaatgagaattactgtcggaatccggacgatgatgctcatggaccctggtgctacacgggcaatccgctcattccttgggattattgccctatttctcgttgtgagggtgataccacgcctacaatagtcaatttagatcatcccgtaatatcatgtgccaaaacgaaacaattgcgagttgtaaatgggattccgacgcgcacaaacatagggtggatggttagtttgagataccgtaataagcatatctgcggaggatcattgatcaaggagagttgggttctgacagctcggcagtgtttcccttctcgggaccttaaggattatgaggcttggcttgggattcatgatgtccacgggaggggagacgagaaatgcaagcaggttctcaatgtttcgcagttggtgtatggtcctgagggatcggatctggtgctgatgaagttggccaggcctgcggtcctggacgattttgttagtacgattgatttgcctaattatggttgcacaattccggagaagaccagttgcagtgtgtatggctggggctacactgggttgatcaactatgatgggctactgcgagtggcacatttgtacataatggggaatgagaagtgctcccagcatcatcgggggaaggtgactctgaatgagtcggaaatatgtgctggggctgagaagattggatcaggaccgtgtgagggggattatggtgggccattggtatgtgagcagcataagatgaggatggttttgggtgtcatcgttccgggtcgtggatgtgctattccgaataggcccggtatatttgtccgagtagcatactacgcgaaatggatacacaaaattatactcacatataaggttccacagtca

SEQ ID NO: 27 - RNA, 2184 nt, synthetic construct
  source 1..2184: mol_type = other RNA
atgtgggtgacaaagctgttgcctgcgttattgctgcagcacgttctgctgcatctgctgctcctccccatagcgataccctatgcggaggggcagcggaagcggcggaacactattcatgagtttaagaagagcgccaagacgactttgataaagatcgatccggcgctcaagattaaaaccaagaaggtgaataccgcagatcagtgcgccaacaggtgcactcgaaacaagggcctgccgttcacgtgcaaggcctttgttttcgacaaggctcgaaagcagtgcctgtggttcccttttaactccatgagcagtggagttaagaaggaattcgggcacgagttcgacctgtacgagaataaagattacatacgtaattgtattattggtaaggggagatcgtacaagggtacggtctccattaccaagtcgggcatcaagtgccaaccgtggagctcgatgatacctcacgagcattcattcctgccctcctcctaccgggggaaggacctgcaggagaattactgcaggaacccccggggggaggagggcgggccgtggtgcttcacgagcaacccggaggttcggtacgaggtgtgcgacatcccgcagtgtagcgaggtcgagtgtatgacatgtaatggcgagtcttaccggggtctgatggaccatacagagtccggtaagatttgccagcggtgggatcatcagacccctcatcggcataagttcctgccggagcgttatcccgataaggggtttgatgataactactgccgcaacccggacgggcagccccgtccgtggtgctatacgctcgacccgcacacgcggtgggagtactgcgctattaagacgtgcgcagacaatacgatgaatgacactgacgtgcccctggagaccacggagtgtattcaggggcagggcgaggggtatcggggaacggtgaacaccatctggaatgggatcccgtgtcagcgctgggactctcagtacccgcatgagcacgacatgaccccggagaactttaagtgtaaggatctccgggagaattattgtcgtaatcctgacgggtctgagagtccctggtgcttcacgacggatcccaatatccgggtgggttactgttcccagatacccaattgcgacatgtcgcatggtcaggactgctaccgggggaatgggaagaattacatgggtaatttatcccagacccggagcggcctgacctgcagcatgtgggataagaatatggaggacctccataggcatatcttttgggagcctgacgccagtaagctcaatgagaattattgtcggaaccccgacgatgatgctcatgggccctggtgttatacgggtaaccccctcataccgtgggattattgtccgatctcgcggtgtgagggggataccacgccgacgattgtcaaccttgaccatcctgtcatttcgtgtgcgaagacaaagcagctgcgggtggttaacgggattccgacccgaaccaacattgggtggatggtctctctccggtatcgtaacaagcatatttgtggcgggtcgctcataaaggagagttgggtcctgacggcccgtcagtgtttccccagccgggacctcaaggattatgaggcctggctggggatacacgacgtgcacgggcggggggacgagaagtgcaagcaggtgcttaacgtctcccagctcgtgtacgggccggagggatccgacttggtgctgatgaagttggccaggccagcggtcctggacgacttcgtcagcaccatcgacctgcccaattatggttgtacgataccggagaagacttcttgttccgtgtacgggtggggctacacggggctcatcaactatgatgggctcctacgtgtggcccacctgtacattatggggaacgagaagtgttctcagcaccatcgcggaaaggtcactctcaatgagagtgagatctgcgctggtgctgagaagattggcagcggcccctgcgagggcgattacggagggcccctcgtctgtgaacagcacaagatgaggatggtcctcggcgtgatcgtccctggcaggggctgcgccatcccgaatcgcccgggtattttcgtccgcgtggcgtactacgccaagtggatacacaagattatcttgacgtataaggttccacagtca

SEQ ID NO: 28 - RNA, 2184 nt, synthetic construct
  source 1..2184: mol_type = other RNA
atgtgggtaacgaaactgttaccagcactactgcttcagcatgtccttctgcatttgttattgctaccaatagcaataccatatgcggaaggacagcgcaaacgtaggaacactatacacgagttcaagaaatccgcgaagaccactctcataaagatcgatcccgcattgaagataaagacaaagaaggtcaacacggccgatcagtgtgccaacaggtgtacgcgcaacaaagggttgccgttcacctgtaaggcttttgtcttcgataaggcgcggaagcagtgcctgtggttcccttttaactccatgtcgagtggagttaagaaggaattcgggcacgagttcgatctttatgagaacaaggattatataagaaattgtataatcgggaaggggcgctcgtacaagggaaccgtctcgatcaccaagtccggcatcaagtgccagccgtggtcgtcgatgatccctcacgagcactccttcctgccctcctcctaccgcgggaaggacctgcaggaaaactactgcaggaacccccgcggcgaggaggggggcccttggtgcttcacctccaacccggaggtgaggtacgaagtctgtgacatcccacagtgttctgaggtcgagtgtatgacttgcaacggcgagtcataccgcggcctcatggatcacacggagtcaggcaagatttgtcagcggtgggatcaccagaccccccaccgtcacaagtttttgccagagcggtaccccgacaagggtttcgacgataactattgtcgaaaccctgacgggcagccccgaccgtggtgctatactctcgacccgcacacgagatgggagtattgcgccatcaaaacctgtgctgacaataccatgaatgacactgacgtgcccttggaaaccacggagtgcatccaagggcagggtgagggttatcgtggtactgtcaacacgatttggaatgggatcccttgtcagcgctgggactctcagtaccctcatgagcacgatatgaccccggagaactttaaatgtaaagatctccgggaaaattactgtcgtaatcctgacggttctgagagtccctggtgcttcacgacggatcccaacatccgggtcgggtactgttctcagattcccaactgtgatatgtcccatggccaggattgctatcggggcaatgggaaaaattacatggggaatctgagtcagacccgatccggactgacttgttccatgtgggacaagaatatggaggacctgcaccgccacatcttttgggagccggacgcttccaaactcaatgagaattattgccggaacccggacgatgatgctcatggcccctggtgctacaccgggaatccattgataccgtgggattactgtcccatctcgcgctgcgagggggacactactcccacgattgttaatctggaccaccctgttattagttgcgctaaaactaaacaattacgtgtagtgaatgggatacccactcgcaccaacattggttggatggtttccctccggtaccgtaacaagcacatttgcggtgggagcctgattaaggagtcttgggtcctgaccgccaggcagtgttttccttcaagggacttgaaggactacgaggcctggctgggcatccacgatgtccatggtcggggcgacgagaaatgtaaacaggttctcaacgtaagtcagcttgtctacggaccggagggatcagatctcgttctcatgaaactagcgcgacctgcggtcctcgatgattttgtgagcactatcgatctccctaattacgggtgcaccatcccggagaagaccagttgtagtgtctacggatggggctacactggcctcattaactacgacggtcttctccgtgtggcgcacctgtacattatggggaacgagaagtgctcacaacatcatcgaggaaaggtcacgctgaatgagagcgagatctgtgcaggagccgagaaaatcggcagtgggccctgcgagggcgactatggtggtccgctcgtgtgcgaacagcacaagatgcggatggtcctgggcgtcattgtccccggccgagggtgtgcaatacccaaccggccggggatattcgtccgggtggcatattatgcgaagtggatccacaagatcatacttacgtataaagtgccacaatcc

SEQ ID NO: 29 - RNA, 2184 nt, synthetic construct
  source 1..2184: mol_type = other RNA
atgtgggttactaagctcctcccagcactgctgctccagcacgtcctcctccatctgttgctcctccccatagcgatcccctatgcggaggggcaacggaagcgccggaacacgatccatgaattcaagaagtctgcaaagactactttaatcaagatcgatccggcgctcaagattaagacaaagaaggtcaacacggccgatcagtgtgctaatcgttgcaccagaaacaaaggcctgccgttcacgtgcaaggcctttgtttttgacaaggccaggaagcagtgcctgtggttcccttttaactccatgtccagtggagttaagaaggaattcgggcacgaatttgatctttatgaaaataaagattacattcgtaactgcatcattggcaaggggcgctcgtacaagggaacggtgagcatcaccaagtcggggatcaagtgccagccctggtcctcgatgattcctcacgagcactccttcctgccttcttcctaccgcgggaaggatctccaggagaattactgccgtaatcctcgtggagaagagggcgggccttggtgcttcacttccaaccctgaggtacgttacgaggtttgtgacatacctcagtgttcggaggtggagtgcatgacttgcaacggcgagtcataccggggtctgatggaccacaccgagtctggcaagatttgccaacggtgggaccatcagactccgcatcggcacaagttcttgccggagcggtaccccgacaagggtttcgacgataactattgtcgaaaccctgacgggcagccgcgaccatggtgctatactctcgacccgcacacgcgttgggagtattgcgccatcaaaacttgtgctgataacaccatgaatgacaccgacgtcccgctcgagacgacggagtgcattcaaggtcagggggaggggtatcgcggcaccgtcaataccatctggaacgggataccgtgccagagatgggacagccagtacccccacgagcatgatatgactccggagaacttcaagtgcaaggatttgcgtgagaattattgccggaatcctgacggttccgagtcaccatggtgtttcacaacggacccgaacatccgggtcgggtactgctctcagattcccaactgtgatatgagtcatggccaggattgctatcggggcaatggcaagaattacatggggaatctgagtcagacccgatccggactgacgtgttcgatgtgggacaagaacatggaggacttgcaccgtcacatattctgggagcctgacgccagcaagctcaatgagaattattgtcggaaccccgacgatgatgctcacgggccctggtgttacacgggtaatcctttgataccgtgggactactgtccgatctcgcgctgcgagggggacactactcccacgattgtcaatctggatcacccggtgatttcttgcgcaaagaccaagcaactccgtgtcgtaaatgggataccgacgaggacgaacatcggctggatggtgtccttgcggtatcgaaacaagcacatctgcggtgggtcgctcatcaaggagagttgggtcctgacggcccgtcagtgtttcccttctcgtgatctgaaagattacgaggcctggctcgggatacacgacgtacacgggcggggagacgagaagtgcaagcaagtacttaacgtctcccagctcgtgtacgggccggagggatccgacttggtcttgatgaagctcgctcgcccagctgtgctcgatgattttgtgagcactatcgatctccctaattacgggtgcaccatcccggagaagacctcttgtagtgtctacggatggggctacactggcctcatcaactacgacggtcttctccgagtggcgcacctgtacattatggggaacgagaagtgctcacaacatcatcgcggcaaggtcactctcaacgagagtgagatttgtgctggagccgagaaaatcggttctggtccatgtgagggtgattatgggggtccactggtatgtgagcaacacaagatgaggatggtcctgggggtcattgttcccggccgagggtgtgccatacccaaccggccggggatcttcgtccgtgttgcttactatgccaagtggatccacaagatcattctcacgtacaaggtgccccagagc

SEQ ID NO: 30 - RNA, 2184 nt, synthetic construct
  source 1..2184: mol_type = other RNA
atgtgggtgaccaagctgttaccagctctgttactgcagcatgtcttgcttcatctcttgctcttgcctatcgccatcccttacgctgagggtcagcgtaagcgtaggaacacgatccatgaattcaagaagtctgcaaagactactttaatcaagatcgatcctgcgctcaagataaagacaaagaaggtcaacacggccgatcagtgtgccaacaggtgcacccgcaacaaggggttgccgttcacctgtaaggcctttgtcttcgacaaggcccgtaagcagtgcctgtggttcccttttaactccatgtccagtggagttaagaaggaattcgggcacgaatttgatctttatgaaaataaagattacattcgtaactgcattatcgggaaggggcgctcgtacaagggaaccgtatcgatcaccaagtctggcatcaaatgccagccttggtcatcgatgattcctcacgagcactccttcctgccttcctcctaccgcgggaaggatctccaggagaattactgccgtaatcctcgtggagaggagggagggccttggtgttttacttcgaaccccgaggtaagatacgaggtgtgcgatatcccgcagtgctcggaggtcgaatgcatgacatgcaacggcgaaagttaccgtggcctgatggatcataccgagagcggtaagatctgtcagcggtgggatcaccagacccctcaccgtcacaaatttttgccggagcggtaccccgacaagggtttcgacgataactattgtcgaaaccctgacgggcagccgcgtccgtggtgctacaccctggatcctcacacccgttgggagtactgcgcaataaagacttgcgcagataacacgatgaacgacaccgacgttcccctggagaccacggagtgcatacaggggcagggggagggctaccgcggcacggtgaataccatttggaatggtattccttgccaacggtgggactcccaatacccccatgagcacgacatgactccagagaacttcaagtgtaaggatctgcgcgagaattattgcaggaaccccgacgggagtgagagtccatggtgtttcactacggacccgaacattcgggttggctactgtagccagatcccgaattgcgacatgagccatgggcaagactgttatcggggtaacggcaagaattacatgggcaacttgtcgcagacccgctccggtctcacatgcagcatgtgggacaagaatatggaggatctccatcgtcacatattctgggagccggatgcgtccaagttgaatgagaattattgccgtaaccccgatgacgatgcccatggcccatggtgctatacgggaaacccactcataccgtgggattactgcccgatctcacggtgtgagggggacaccaccccaaccattgttaatctggatcaccctgtcattagctgtgcgaagactaagcagcttcgtgtggttaatggcatcccgacccggactaacattggttggatggtgtctctcagatatcgcaacaagcatatctgtgggggatctcttatcaaggaaagttgggtcctcacggcccgtcagtgtttcccttctcgtgatctgaaagattacgaggcctggctcgggatacacgacgtacacgggcggggagacgagaagtgcaagcaagtacttaacgtctcccagctcgtgtacgggcctgagggttctgacctggtactaatgaagctggcgcggccagctgtattggacgacttcgtcagcaccatcgatttgccaaattatggctgcactatccctgagaagacatcctgcagtgtctacggttgggggtatacggggctcatcaactatgatgggctcctacgcgtggcacacctatacattatgggtaacgagaaatgttctcagcaccaccgcgggaaggtcactctcaacgagagtgagatctgtgctggtgctgagaagattggctcgggtccctgcgagggtgattacggaggacctcttgtctgtgagcaacacaagatgcggatggtcctcggggtgatcgtccctggcaggggctgtgccattcccaaccgtcccggtatttttgtccgtgtggcgtactacgccaaatggatacataagatcatcctgacttataaagtaccacagagc

SEQ ID NO: 31 - RNA, 2184 nt, synthetic construct
  source 1..2184: mol_type = other RNA
atgtgggtgaccaagttgctgccggcgctgttgttgcagcatgttctgctgcatctgctgctcctccccatcgcgatcccgtatgcggaggggcagcggaagcggcggaacacgatccatgagttcaagaagtctgcgaagactacgttgatcaagatcgatccggcgttgaagatcaagaccaagaaggtcaacacggcggatcagtgcgccaaccggtgcactcggaacaaggggctgccgttcacgtgtaaggcctttgttttcgacaaggctcggaagcagtgcctgtggttcccttttaactccatgagcagtggggttaagaaggaattcgggcatgagttcgacctgtacgagaacaaggactatatacggaactgcatcatcgggaagggcaggtcctacaagggcacggtgagcatcacgaagagcggcatcaagtgccagccttggagcagcatgataccgcacgagcacagcttcctgccctcctcctaccgggggaaggacctgcaggagaactactgcaggaacccccggggggaggagggcgggccttggtgcttcacctccaacccggaggtgaggtacgaggtgtgcgatatcccgcagtgctcagaggtggagtgtatgacttgcaacggcgagtcgtacaggggtctgatggaccacaccgagagtggcaagatttgccagcggtgggaccatcagaccccccacaggcacaagttcctgccggagcgctacccggacaagggtttcgacgataactattgtcggaacccggacgggcagccccggccgtggtgctacactcttgatccgcatacccggtgggagtactgcgcgatcaagacgtgcgcggacaacaccatgaacgataccgacgtccccctggagaccactgagtgcattcagggccagggggaggggtatcggggtactgtgaacaccatctggaacgggatcccgtgccagaggtgggactcgcagtacccccacgagcacgacatgaccccggagaactttaagtgtaaggatctccgggagaattactgtcgtaatcctgacgggagtgagagcccctggtgcttcacgacggatccgaacatccgcgtggggtattgcagtcagatccctaactgcgacatgagccatgggcaggattgttatcggggtaacggcaagaattacatgggcaacttgtcgcagacgcgctccggtctcacgtgcagcatgtgggacaagaatatggaggatctgcatcgtcacatattctgggagccggatgcgagcaagttgaacgagaattattgccgtaaccccgatgacgacgcccatggcccgtggtgctacactggtaaccccctcataccgtgggattactgcccgatctcgcggtgtgagggggataccacgcccacgatcgttaatctggaccaccccgtgatatcttgtgcgaagacgaagcagctccgtgtggtcaatggtattccgacgcggactaacattggctggatggtgtcgcttcgttatcgcaacaagcatatctgcggggggtcgctgattaaggagtcgtgggtgctgacggcgaggcagtgcttcccgtcgcgtgatctgaaggattacgaggcctggctgggcatccacgatgtccacgggcgtggggatgagaagtgtaagcaggtgcttaacgtttctcagctcgtgtacggcccggagggctctgacctcgtgttgatgaagttggcccgccctgccgttctcgatgactttgtttcgactatcgatctccctaattacgggtgcaccatcccggagaagaccagctgtagcgtctacggctggggctatacgggtttgataaactacgatggccttctccgggtggcgcacctgtacattatggggaacgagaagtgttctcagcaccaccgcgggaaggtcactctcaacgagagtgagatctgcgctggtgctgagaagatcgggtcggggccctgcgagggcgactacgggggaccgctggtctgcgagcagcataagatgaggatggtcttgggggtgattgtccccggccgggggtgtgcgatacccaaccggccggggatattcgtccgcgtggcgtactacgccaagtggatccacaagatcatcctcacttataaggtgccgcagtcg

SEQ ID NO: 32 - RNA, 2478 nt, synthetic construct
  source 1..2478: mol_type = other RNA
taatacgactcactataaggaaataagagagaaaagaagagtaagaagaaatataagagccaccatgtgggtaacaaaattgctacctgcattgctgctacagcatgttctgctgcatctgctgctcctccccatagcgatcccctatgcggaggggcagcggaagcggcggaacaccatacatgagtttaagaagtctgcgaagactactctcatcaaaatcgatcccgccctgaagataaagacgaagaaggtgaataccgcagaccagtgcgcgaaccgttgtacgaggaacaaaggcctgccgttcacgtgcaaggcctttgtcttcgacaaggctcgcaagcagtgtctgtggtttcccttcaactctatgtcttcaggggtgaagaaggaattcggacacgaattcgatctttacgaaaacaaggactatatacgtaattgtataattgggaaggggcgctcgtataagggaacagtctcgatcacgaagtctggcataaagtgccagccgtggtcgagtatgatccctcacgagcactccttcctgccctcctcctaccgggggaaagacctgcaggaaaattactgcaggaacccccggggggaggagggcggtccttggtgcttcacctccaaccctgaggtgcgttacgaagtttgtgacatacctcagtgttcggaggtggagtgcatgacctgtaacggggagtcttaccggggtctgatggaccacaccgagtctggcaaaatttgccagcggtgggaccatcagaccccgcaccggcataaatttttacctgagcggtaccccgataagggtttcgacgataactattgtcgaaaccctgacgggcagccgcgtccgtggtgttacacgttggatccgcacacgcgatgggagtactgtgcaatcaagacgtgtgcggataatacgatgaacgatacggacgtgcccctggagaccacggagtgtatccaggggcagggtgaagggtatcgggggacagtgaacaccatctggaatgggatcccgtgtcagcgctgggactctcagtacccgcatgagcacgacatgaccccggagaactttaaatgtaaagatctccgggagaattactgtcgtaatcctgacgggtctgagagtccctggtgcttcacgacggatcccaacatccgggtgggttactgttcccagatacccaactgtgacatgagtcacggtcaggattgttatcggggtaacggcaagaattacatgggcaacttgtcgcagacgcgctccggtctcacatgcagcatgtgggacaagaatatggaggatctacatcgtcacatattctgggagccggatgcgagcaagttgaacgagaattattgccgtaaccccgatgacgatgctcatggtccctggtgttatacgggaaacccgttgataccatgggactattgcccgatttctcgttgcgagggggatacgacgcccacgatcgttaatctggaccaccccgtgatatcttgtgcgaagacgaagcagctccgtgtagtcaatgggattccgacacggactaacattggctggatggtgtcgcttcgttatcgcaacaagcatatctgcggggggtccttgattaaggaatcgtgggtgctgacggcccgtcagtgtttccccagccgggacctcaaggattatgaggcctggctggggatacacgacgtacacgggcggggggacgagaagtgcaaacaagtacttaacgtctcccagctcgtgtacgggccggagggatcagatctcgttctcatgaaattagcgcgacctgctgtcctcgatgattttgtgagcactatcgatctccctaattacgggtgcaccatcccggagaagaccagttgtagtgtatatggatggggctacactggcctcatcaactacgacggtcttctccgggtggcgcacctgtatattatggggaacgagaagtgctcacaacatcatcgaggcaaggtcacgctgaatgagagcgagatctgcgcaggggccgagaagataggttccggcccctgcgagggggactacggtggtccccttgtatgtgagcagcataagatgaggatggtcttgggagtgattgtccccggccgggggtgtgcaatacccaaccggccggggatattcgtccgcgtggcgtactacgccaagtggatccacaagatcatcctcacttataaggttccacagtcctgataataggctggagcctcggtggccatgcttcttgccccttgggcctccccccagcccctcctccccttcctgcacccgtacccccgtggtctttgaataaagtctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - RNA, 2635 nt, synthetic construct
  source 1..2635: mol_type = other RNA
taatacgactcactataaggaaataagagagaaaagaagagtaagaagaaatataagagccaccatgtgggtgacaaagttgttaccggccctgttacttcagcatgtcttgcttcacttgcttctgttgccgatcgccattccttacgctgagggtcagcgtaagcgccggaacacgatccatgaattcaagaagtctgcgaagactaccttgatcaagatcgatccggcgctaaaaatcaagactaagaaggtgaataccgcagaccagtgcgcgaaccgttgtacgaggaacaagggcctgccgttcacgtgcaaggcctttgtcttcgacaaggctcgcaagcagtgtctgtggtttcccttcaatagtatgagctcgggggtcaagaaggagttcgggcatgagttcgatctctacgagaacaaggactacatccggaactgcatcattgggaaggggcgctcgtacaaggggaccgtgtcgatcacgaagtctggcatcaagtgccagccgtggtcgagcatgatccctcacgagcactccttcttgccctcgagctaccgcgggaaggacctgcaggagaattactgcaggaacccccgcggtgaggagggcggtccatggtgcttcaccagcaatccggaggtcaggtacgaggtatgcgacatacctcagtgctctgaggtggagtgcatgacatgtaacggcgagagttacaggggcctgatggatcataccgaatccggcaagatttgtcagcggtgggatcaccagactccgcaccgccacaagttcttgccggagcggtatccggataagggtttcgacgataactattgtcgaaaccctgacggtcagcctcgtccatggtgctacacgcttgatccgcatacccggtgggagtactgcgcgatcaagacgtgtgccgataacaccatgaacgacactgacgtcccattggagacaaccgagtgcatacagggacagggcgagggttatcggggtactgtgaacaccatctggaacgggatcccgtgccagaggtgggattcacagtacccccacgagcatgacatgactcctgagaatttcaagtgcaaggatctccgagagaactactgtcggaacccggacggttcggagtcgccgtggtgcttcaccacggacccgaacatccgggtggggtactgttcgcagatccccaactgcgacatgtctcacgggcaggactgttaccgggggaacgggaagaactatatgggcaacctgtcccagacacggagcgggctaacctgctccatgtgggataagaatatggaggacctccataggcatatcttctgggagccggatgcctctaagctcaatgagaattattgccggaacccggatgatgatgctcatgggccgtggtgttatacggggaacccgttgataccgtgggactactgtcccatctcgcgatgcgagggggacactactcccactatagttaacctggaccacccggtgatatcctgcgcgaagacaaagcagttgcgggtcgtgaacggtatccccacccggaccaacataggttggatggtcagcctccgctacagaaataagcatatctgtggcgggtcgctgatcaaggagtcatgggttctgacagccaggcagtgctttccgtcccgcgacttgaaggactacgaggcctggttgggcattcatgacgtacacgggcggggagacgagaagtgcaagcaagtacttaacgtctcccagctcgtgtacggtcctgaagggtccgacctggtcttgatgaagctcgcccgccctgctgtattggatgacttcgtgtcaaccatagatttgccgaactatggttgcacgatccctgagaagactagttgctcggtgtacgggtggggctacacagggctcatcaactatgatgggctgctgcgcgtagcccacctgtacatcatgggcaacgagaagtgtagccagcatcaccggggcaaggtcactctcaacgagagtgagatttgcgccggtgccgagaaaatcggcagcggcccctgcgagggcgattacggagggccgctcgtctgtgagcagcacaagatgaggatggtcctcggggtgatcgtgcctggcaggggctgcgcgatacctaatcgtccggggatcttcgtccgcgtggcgtactacgccaagtggatacataagatcatcctgacgtataaggtaccgcagagctgagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 34 - RNA, 2468 nt, synthetic construct
  source 1..2468: mol_type = other RNA
taatacgactcactataaggaaataagagagaaaagaagagtaagaagaaatataagagccaccatgtgggtgaccaagctcctgccggccctgctgctccagcatgtccttctgcatctcctcctgctacccatagcgatcccctatgcagaaggacaaaggaaacgcagaaatacaattcatgaattcaagaaatcagcgaagactaccctaatcaagatagatccagcactgaagataaaaaccaagaaagtgaataccgcagaccagtgtgctaaccgttgtacgaggaacaaaggactgccattcacttgcaaggcttttgtttttgataaggcccgtaaacaatgcttgtggttccccttcaacagcatgtcaagtggcgtgaagaaagagttcggacacgaatttgacctctatgagaacaaagactacattaggaactgcatcatcggtaagggacgctcgtataagggcacagtttctatcactaagagtggcattaaatgtcagccctggtcgtccatgataccacacgaacacagctttcttccttcttcctatcgcggaaaggacttgcaggaaaactactgtcgcaatccgcgaggggaagaagggggaccctggtgcttcacaagcaaccctgaggtacgctacgaagtctgtgacattcctcagtgttccgaagttgaatgcatgacctgcaacggggagtcgtatcgaggtctcatggatcacacagaatcaggcaagatttgtcagcggtgggatcaccagaccccccatcgccacaaattcttgcctgaacgataccctgacaagggttttgatgataattactgccgtaaccccgacggccagccgaggccctggtgttacactcttgaccctcacacccgatgggagtactgtgctattaagacgtgcgcggacaatactatgaatgacactgatgtgcctttggagacaactgagtgcatacaaggtcaaggcgaaggctaccggggtactgtgaatacaatttggaatgggataccatgtcagagatgggattcgcagtaccctcacgagcatgacatgactcctgaaaatttcaagtgcaaggacctacgagagaattactgccgaaatccagatgggtctgagagcccctggtgctttaccactgatccgaacatcagagttggttactgctcccaaataccaaactgtgatatgtcgcacggacaagattgctatcgggggaatggcaaaaactacatgggcaacctgagtcaaacaagatctggactaacatgttctatgtgggacaagaacatggaagatcttcatcgtcatatcttctgggaaccggatgcaagtaagctgaacgagaattactgcagaaatccagacgatgatgctcacggaccctggtgctacacgggaaatccactcattccttgggattactgccccatttctcgttgtgaaggtgacaccacacctaccatagtcaacctggaccatcccgttatatcatgtgccaaaacgaaacaattgcgagttgtcaatgggatcccaactcgaactaacatcggatggatggtttccctcagataccgtaacaaacatatctgcgggggatcattgatcaaggagagttgggttcttacggcaaggcagtgtttcccttcgcgagacttgaaggattacgaagcttggcttggaattcacgatgtccacggaagaggagatgagaaatgcaaacaggttctcaatgtttcgcagcttgtatatggcccggaaggatcagatctggtgttaatgaagttagccaggccggccgtcctggatgatttcgttagtacaatcgatcttcccaattatggttgcacaatcccggagaagaccagttgtagcgtctatggctggggctacactggattgatcaactatgatgggctattacgagtggcacatctctatataatgggaaatgagaaatgctcgcagcatcaccgagggaaggtgactctgaacgagtcggaaatatgtgctggggccgagaagattggttctggcccatgtgagggggattacggtggcccactggtttgtgagcaacacaaaatgaggatggttcttggtgttattgttcctggtcggggatgtgccattccaaaccgtcctggtatttttgtccgtgtggcatattacgcaaaatggatacacaagattattctcacctataaggtaccccagtcatgataataggctggagcctcggtggccatgcttcttgccccttgggcctccccccagcccctcctccccttcctgcacccgtacccccgtggtctttgaataaagtctgaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2468 nt, synthetic construct
  source 1..2468: mol_type = other RNA
taatacgactcactataaggaaataagagagaaaagaagagtaagaagaaatataagagccaccatgtgggtgaccaagctgttaccagctctgttactgcagcatgtcttgcttcatctcttgctcttgcctatcgccatcccttacgctgagggtcagcgtaagcgtaggaacacgatccatgaattcaagaagtctgcaaagactactttaatcaagatcgatcctgcgctcaagataaagacaaagaaggtcaacacggccgatcagtgtgccaacaggtgcacccgcaacaaggggttgccgttcacctgtaaggcctttgtcttcgacaaggcccgtaagcagtgcctgtggttcccttttaactccatgtccagtggagttaagaaggaattcgggcacgaatttgatctttatgaaaataaagattacattcgtaactgcattatcgggaaggggcgctcgtacaagggaaccgtatcgatcaccaagtctggcatcaaatgccagccttggtcatcgatgattcctcacgagcactccttcctgccttcctcctaccgcgggaaggatctccaggagaattactgccgtaatcctcgtggagaggagggagggccttggtgttttacttcgaaccccgaggtaagatacgaggtgtgcgatatcccgcagtgctcggaggtcgaatgcatgacatgcaacggcgaaagttaccgtggcctgatggatcataccgagagcggtaagatctgtcagcggtgggatcaccagacccctcaccgtcacaaatttttgccggagcggtaccccgacaagggtttcgacgataactattgtcgaaaccctgacgggcagccgcgtccgtggtgctacaccctggatcctcacacccgttgggagtactgcgcaataaagacttgcgcagataacacgatgaacgacaccgacgttcccctggagaccacggagtgcatacaggggcagggggagggctaccgcggcacggtgaataccatttggaatggtattccttgccaacggtgggactcccaatacccccatgagcacgacatgactccagagaacttcaagtgtaaggatctgcgcgagaattattgcaggaaccccgacgggagtgagagtccatggtgtttcactacggacccgaacattcgggttggctactgtagccagatcccgaattgcgacatgagccatgggcaagactgttatcggggtaacggcaagaattacatgggcaacttgtcgcagacccgctccggtctcacatgcagcatgtgggacaagaatatggaggatctccatcgtcacatattctgggagccggatgcgtccaagttgaatgagaattattgccgtaaccccgatgacgatgcccatggcccatggtgctatacgggaaacccactcataccgtgggattactgcccgatctcacggtgtgagggggacaccaccccaaccattgttaatctggatcaccctgtcattagctgtgcgaagactaagcagcttcgtgtggttaatggcatcccgacccggactaacattggttggatggtgtctctcagatatcgcaacaagcatatctgtgggggatctcttatcaaggaaagttgggtcctcacggcccgtcagtgtttcccttctcgtgatctgaaagattacgaggcctggctcgggatacacgacgtacacgggcggggagacgagaagtgcaagcaagtacttaacgtctcccagctcgtgtacgggcctgagggttctgacctggtactaatgaagctggcgcggccagctgtattggacgacttcgtcagcaccatcgatttgccaaattatggctgcactatccctgagaagacatcctgcagtgtctacggttgggggtatacggggctcatcaactatgatgggctcctacgcgtggcacacctatacattatgggtaacgagaaatgttctcagcaccaccgcgggaaggtcactctcaacgagagtgagatctgtgctggtgctgagaagattggctcgggtccctgcgagggtgattacggaggacctcttgtctgtgagcaacacaagatgcggatggtcctcggggtgatcgtccctggcaggggctgtgccattcccaaccgtcccggtatttttgtccgtgtggcgtactacgccaaatggatacataagatcatcctgacttataaagtaccacagagctgataataggctggagcctcggtggccatgcttcttgccccttgggcctccccccagcccctcctccccttcctgcacccgtacccccgtggtctttgaataaagtctgaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa